(19) United States
(12) Patent Application Publication
(10) Pub. No.: US 2003/0134865 A1
(43) Pub. Date: Jul. 17, 2003

(54) Modulation of histone deacetylase

(76) Inventors: Ian Adcock, London (GB); Samson Lim, New South Wales (AU); Kazuhiro Ito, Tokyo (JP); Peter John Barnes, London (GB)

Correspondence Address: C G Mersereau, Nikolai & Mersereau, 820 International Centre, 900 Second Avenue South, Minneapolis, MN, 55402-3325, US

(21) Appl. No.: 10/220,342

(22) PCT Filed: Mar. 2, 2001
(86) PCT No.: PCT/GB01/00905

(51) Int. Cl. A61K 31/522; G01N 33/53

(52) U.S. Cl. 514/263.34; 514/263.35; 435/7.1

(57) ABSTRACT

A screening method for identifying a drug-like compound or lead compound for the development of a drug-like compound in which (1) a xanthine or related compound is exposed to a histone deacetylase, (2) the binding of the compound to the histone deacetylase is measured or the change in the activity of the histone deacetylase is measured or the change in the binding of the histone deacetylase to activated glucocorticoid receptor (GR) is measured and (3) any compound capable of the required binding to the histone deacetylase or producing the required change in the activity of the histone deacetylase or its binding to activated glucocorticoid receptor is identified. Methods of treatment make use of compounds identifiable by the screening methods.

The present invention relates to the modulation of histone deacetylase activity by small molecules.

Recent advances have elucidated the mechanisms for the control of gene transcription and how corticosteroids may suppress the expression of multiple inflammatory genes (Barnes, P. J. (1998) Clin. Sci. (Colch.) 94:557-572). In the resting cell, DNA is tightly compacted to prevent transcription factor accessibility. During activation of the cell this compact, inaccessible DNA is made available to DNA-binding proteins, thus allowing the induction of gene transcription (Beato, M. (1996) J. Mol. Med. 74:711-724; Wolffe, A. P. (1997) Nature 387:16-17). DNA is packaged into chromatin, a highly organised and dynamic protein-DNA complex. The fundamental subunit of chromatin, the nucleosome, is composed of an octamer of 4 core histones; an H3/H4 tetramer and two H2A/H2B dimers, surrounded by 146 bp of DNA (Beato, M (1996) J. Mol. Med. 74:711-724; Beato, M. & K. Eisfeld (1997) Nucleic. Acids. Res. 25:3559-3563). The packaging of DNA into nucleosomes acts as a barrier to the initiation of transcription by preventing the access of transcription factors, and RNA polymerase II, to their cognate recognition sequences (Workman, J. L. & A. R. Buchman (1993) Trends. Biochein. Sci. 18:90-95). The N-terminal tails of the core histones contain highly conserved lysines that are sites for post-transcriptional acetylation. In addition, core histones may be modified by phosphorylation, methylation, ADP ribosylation or ubiquitinisation of specific amino acid residues (Wu, R. S et al (1986) CRC Crit. Rev. Biochem. 20:201-263). Histone acetylation is thought to be a dynamic process which occurs on actively transcribed chromatin only (Perry, M. & R. Chalkley (1982) J. Biol. Chem. 257:7336-7347). Histone-4 is the most important for transcriptional regulation (Imhof, A. & A. P. Wolffe (1998) Curr. Biol. 8:R422-R4248). Acetylation of histones by co-activator proteins such as CREB-binding protein (CBP) facilitates transcription.

There is compelling evidence that increased gene transcription is associated with an increase in histone acetylation, whereas hypo-acetylation is correlated with reduced transcription or gene silencing (Ura, K et al (1997) EMBO J. 16:2096-2107; Wolffe, A. P (1997) Nature 387:16-17). Targeted acetylation of histone H4 tails plays an important role in allowing regulatory proteins to access DNA and is likely to be a major factor in the regulation of gene transcription (Lee, D. Y et al (1993) Cell 72:73-84; Nightingale, K. P et al (1998) EMBO J. 17:2865-2876; Rundlett, S. E et al (1998) Nature 392:831-835).

Glucocorticoids are the most effective therapy for the treatment of inflammatory diseases such as asthma, a chronic inflammatory disease of the airway (Barnes, P. J (1998) Clin. Sci. 94:557-572). Functionally, they act partly by inducing some anti-inflammatory genes such as secretary leukocyte proteinase inhibitor (SLPI) (Sallenave, J. M et al (1994) Am. J. Respir. Cell Mol. Biol. 11:733-741), Lipocortin-1 (Flower, R. J. & N. J. Rothwell (1994) Trends. Pharmacol. Sci. 15:71-76) and IL-1 receptor antagonist (Levine, S. J et al (1996) Am. J. Respir. Cell Mol. Biol. 15:245-251) but mainly by repression of inflammatory genes, such as cytokines, adhesion molecules, inflammatory enzymes and receptors (Barnes, P. J (1998) Clin. Sci. 94:557-572). They are thought to act by binding to a cytosolic glucocorticoid receptor (GR), which upon binding is activated and rapidly translocates to the nucleus. Within the nucleus, GR either induces gene transcription by binding to specific DNA elements in the promoter/enhancer regions of responsive genes or reduces gene transcription by transrepression (Truss, M. & M. Beato (1993) Endocr. Rev. 14:459-479). GR reduces gene transcription by interaction with pro-inflammatory transcription factors such as AP-1 and NF-κB (Barnes, P. J. & I. M. Adcock (1998) Eur. Respir. J. 12:221-234.2; Ray, A. & K. E. Prefontaine (1994) Proc. Natl. Acad. Sci. U.S.A. 91:752-756; Truss, M. & M. Beato (1993) Endocr. Rev. 14:459-479). Both AP-1 or NF-κB and GR mutually repress each other's ability to activate transcription (Jonat, C et al (1990) Cell 62:1189-1204) and require the co-activator CREB binding protein (CBP) for maximal activity (Gerritsen, M. E et al (1997) Proc. Natl. Acad. Sci. U.S.A. 94:2927-2932; Kamei, Y et al (1996) Cell 85:403-414; Perkins, N. D et al (1997) Science 275:523-527. This suggests that reduction of gene expression by GR may involve interference with transactivation mediated by co-activators such as CBP (Sheppard, K. A et al (1998) J. Biol. Chem. 273:29291-29294) possibly due to competition (squelching) for limiting amounts of the CBP (Kamei, Y et al (1996) Cell 85:403-414). Plesko, M. M et al (1983) J. Biol. Chem. 258:13738-13744 suggests that sodium butyrate, a histone deacetylase inhibitor, may interfere with GR-activated transcription. Many of the above studies rely on the overexpression of components of these pathways, which is generally understood to make interpretation of the studies difficult.

Xanthines, for example theophylline, have been used in the treatment of asthma for over 70 years, but their use has recently declined as inhaled corticosteroids have become the mainstay of asthma control. Furthermore, inhaled β₂-agonists are more effective bronchodilators and their side effects, such as nausea and headaches, commonly occur at previously recommended doses. Originally, theophylline, for example, was considered to be a bronchodilator and the optimal plasma concentrations that gave maximal bronchodilatation with least risk of side effects was found to be 10-20 mg/L (55-110 μM). Theophylline has also been used as a bronchodilator in the treatment of COPD (Chronic obstructive pulmonary disease). Since theophylline is a relatively weak bronchodilator and side effects are relatively common at bronchodilator doses, it has largely been superseded by inhaled β₂-agonists. However, there is increasing evidence that theophylline has a beneficial effect in asthma control that is not explained by bronchodilatation (Barnes, P. J. & R. A. Pauwels (1994) Eur. Respir. J. 7:579-591). Low doses of theophylline, which give a plasma concentration of 5-10 mg/L, improve asthma control. In two large carefully controlled studies low dose theophylline achieved comparable control of asthma to a low dose of inhaled corticosteroids in both children and adults (Reed, C. E et al (1998) J. Allergy Clin. Immunol. 101:14-23; Tinkelman, D. G et al (1993) Pediatrics 92:64-77). In asthmatic patients low dose theophylline reduces inflammatory markers (Jaffar, Z. H et al (1996) Eur. Respir. J. 9:456-462; Ward, A. J et al (1993) Am. Rev. Respir. Dis. 147:518-523), inhibits the eosinophilia induced by inhaled allergen ( Sullivan, P et al (1994) Lancet 343:1006-1008) and reduces the expression of cytolines, such as interleukin(IL)-5 (Finnerty, J. P et al (1996) Eur. Respir. J. 9:1672-1677). Theophylline also reduces the stimulated release of GM-CSF from activated eosinophils in vitro (Shute, J. K et al (1998) Clin. Exp. Allergy 28 Suppl 3:47-52). Long-term treatment with theophylline reduces airway hyperresponsiveness to methacholine challenge (Page, C. P et al (1998) Eur. Respir. J. 12:24-29). In addition, in patients with severe asthma who are withdrawn from theophylline, there is a deterioration of asthma control, despite the fact that patients are maintained on high doses of inhaled corticosteroids (Brenner, M et al (1988) Clin. Allergy 18:143-150; Kidney, J et al (1995) Am. J. Respir. Crit. Care Med. 151:1907-1914). This is associated with an increase in the number of inflammatory cells, particularly CD4⁺ T-lymphocytes, in the airways (Kidney et al (1995)). These studies suggest that low doses of theophylline have anti-inflammatory or immunomodulatory actions in asthma.

Several studies have demonstrated an interaction with corticosteroid therapy and the steroid-sparing effects of theophylline (Lim, S et al (1998) American Journal of Respiratory and Critical Care Medicine 157:A415). In patients with moderate asthma, which was not controlled on budesonide 800 μg daily, addition of low dose theophylline (mean plasma concentration ^{˜}8 mg/L) gave a greater improvement in asthma control, measured as lung function, symptoms and rescue β₂-agonist use, than doubling the dose of inhaled corticosteroid (Evans, D. J et al (1997) N. Engl. J. Med. 337:1412-1418.4). Similar results were obtained in patients with milder asthma (Lim et al (1998); Ukena, D et al (1997) Eur. Respir. J. 10:2754-2760). These studies suggest that there may be a beneficial interaction between low dose theophylline and corticosteroid in the long-term control of asthma and that theophylline has a molecular mechanism of action that differs from that of corticosteroids. This may be exploited in the control of severe asthma, when addition of theophylline may improve asthma control despite the fact that high doses of inhaled or oral corticosteroid are used (Rivington, R. N et al (1995) Am. J. Respir. Crit. Care Med. 151:325-332).

There remains uncertainty about the molecular mechanisms for the anti-inflammatory action of xanthines, for example theophylline. There is convincing evidence that the bronchodilator action of theophylline can be explained by inhibition of phosphodiesterases (PDEs; chiefly PDE3 and PDE4) in airway smooth muscle (Rabe, K. F et al (1995) Eur. Respir. J. 8:637-642). However, this is unlikely to account for the anti-inflammatory or immunomodulatory effects of theophylline, since the inhibitory effect of theophylline on PDE activity is trivial at concentrations of <50 μM. Another proposed mechanism involves antagonism of adenosine receptors, since adenosine is a bronchoconstrictor in asthma and adenosine receptor antagonism may occur at therapeutic concentrations. Some of the serious side effects of theophylline, including cardiac arrhythmias and seizures may be due to adenosine receptor antagonism.

There is also evidence for other anti-inflammatory mechanisms that cannot be accounted for by either PDE inhibition or adenosine receptor antagonism. A recent study showed that low concentrations of theophylline were able to inhibit the activation of the transcription factor nuclear factor-κB (NF-κB) and thus to reduce the expression of inflammatory genes in a manner similar to corticosteroids (Tomita, K et al (1999) Naunyn Schmiedebergs Arch. Pharmacol. 359:249-255). Other studies have demonstrated that low concentrations of theophylline decrease survival of eosinophils induced by IL-5 and granulocyte-macrophage colony stimulating factor (GM-CSF) and that this is independent of PDE inhibition and changes in cyclic AMP (Ohta, K et al (1996) Clin. Exp. Allergy 26 Suppl 2:10-15; Yasui, K et al (1997) J. Clin. Invest. 100:1677-1684). The molecular basis for these anti-inflammatory effects is not yet known.

We demonstrate that dexamethasone shows a different pattern of histone H4 acetylation from that seen with IL-1β and at low concentrations (10⁻¹⁰ M) represses IL-1β-stimulated histone acetylation. This does not appear to involve induction of HDAC protein or activity or squelching of CBP. The mechanism of GR repression of IL-1β-stimulated histone H4 K8 and K12 acetylation appears to be by direct inhibition of CBP-associated HAT activity and by active recruitment of a histone deacetylase (HDAC2) complex. The recruited HDAC complex then deacetylates the acetylated histones thereby suppressing inflammatory genes.

We show both in vitro and in vivo that a xanthine, for example theophylline, enhances HDAC activity in epithelial cells. This increased HDAC activity appears to then be available for corticosteroid recruitment and suggests a co-operative interaction between corticosteroids and stimulators of HDAC activity, for example xanthines, for example theophylline. This mechanism occurs at therapeutic concentrations of, for example, theophylline and is dissociated from phosphodiesterase (PDE) inhibition (the mechanism of bronchodilatation) or blockade of adenosine receptors, which are responsible for side effects of theophylline. Thus we have shown that a stimulator of HDAC activity, for example a xanthine, for example theophylline, exerts a novel anti-asthma effect through increasing HDAC activation which is subsequently recruited by corticosteroids to suppress inflammatory genes. Further, theophylline can enhance dexamethasone actions under conditions of oxidative stress where dexamethasone is only weakly effective. This may be very important in severe asthma and COPD where steroids are clinically not effective at doses that do not produce side-effects. Thus xanthines, for example theophylline, may also be steroid-sparing and enhance steroid-responsiveness in these types of patients. The invention further provides associated screening methods and methods of treatment.

Theophylline, theobromine and caffeine are examples of xantiines, in particular methylxanthines. Xanthines have numerous biological activities, as discussed above and, for example, in Martindale: The Extra Pharmacopoeia 32^{nd} Edition, but can be difficult drugs to use because of the spectrum of activities, leading to unwanted effects, and pharmacokinetic profiles that can vary widely between individuals, making it difficult to judge the correct dosage to use for a particular individual. The structure of xanthine is shown in FIG. 18.

It was not appreciated that xanthines could be useful lead compounds for the development of compounds that are selective modulators, in particular activators, of histone deacetylase activity. The present work surprisingly shows that histone deacetylases may be modulated, for example activated, by xanthine compounds. These findings may allow identification and development of histone deacetylase modulators, for example activators, that may be useful as therapeutic agents. The new knowledge concerning the histone deacetylase modulatory effects of xanthines, for example theophylline, may allow use of known xanthines, for example theophylline, for different purposes, for example exploiting the modulation, particularly activation of histone deacetylase.

Histone deacetylases are reviewed, for example, in Johnson & Turner (1999) “Histone deacetylases: complex transducers of nuclear signals” Semin Cell Dev Biol 10, 179-188. WO97/35990 describes histone deacetylases and uses thereof and is hereby incorporated by reference. The following Genbank records are examples of those that relate to human histone deactetylases (HDACs):

| HDAC No | Accession numbers (human) |
|---|---|
| 1 | NP004955, Q13547 |
| 2 | NP001518 |
| 3 | MP003874, AAC26509 |
| 4 | AAD29046 |
| 5 | AAD290487, NP005465 |
| 6 | AAD29048, NP006035 |
| 7 | AAF04254 |

There are 7 HDACs now recognised in mammalian cells and we have found that HDAC1, HDAC2 and HDAC3 are present in epithelial and inflammatory cells.

Methods of preparing and assaying histone deacetylase activity are well known to those skilled in the art and are described in the Examples and references therein, incorporated herein by reference. Histone deacetylases appear to be involved in the modulation of many biological processes, and may be implicated in pathogenic conditions including defects in cellular proliferation and differentiation and in control of gene expression, as discussed, for example, in WO97/35990. Trichostatin A and trapoxin inhibit histone deacetylase activity (see, for example, Yoshida et al (1990) “Potent and specific inhibition of mammalian histone deacetylase both in vivo and in vitro by trichostatin A” J Biol Chem 265, 17174-17179) but specific small-molecule activators of histone deacetylase have not previously been characterised.

Histone deacetylase modulators, in particular inhibitors, have been suggested to be useful in the treatment of various diseases or conditions in WO97/35990 but no evidence of efficacy is presented in any of the diseases or conditions. There is no mention of xanthine derivatives. The diseases or conditions appear to have been selected as those in which there are defects in cellular differentiation and proliferation, for example cancer. There is no mention of asthma.

There is nothing in the prior art to suggest that modulation, particularly activation, of histone deacetylase activity would be useful in the treatment of asthma or other inflammatory airway disease, for example COPD, nor that treatment with such modulators together with corticosteroid treatment would be useful.

There is nothing in the prior art to suggest that xanthines and related compounds would be useful as modulators of histone deacetylase activity.

A first aspect of the invention is a screening method for identifying a drug-like compound or lead compound for the development of a drug-like compound in which (1) a xanthine or related compound is exposed to a histone deacetylase, (2) the binding of the compound to the histone deacetylase is measured or the change in the activity of the histone deacetylase is measured or the change in the ability of the histone deacetylase to bind to activated glucocorticoid receptor (GR) is measured and (3) any compound capable of the required binding to the histone deacetylase or producing the required change in the activity of the histone deacetylase or its ability to bind to activated glucocorticoid receptor is identified.

The purpose of the screen is to identify (and select for further investigation) compounds which may be useful as modulators of histone deacetylase activity. The condition (ie the required binding to the histone deacetylase or required change in the ability of the histone deacetylase to bind to activated glucocorticoid receptors) which the compound must satisfy in order to be identified as a drug-like compound or lead compound for the development of a drug-like compound may be set at a value (expressed, for example, as a binding or dissociation constant) achieved by compounds capable of achieving the required change in the activity of the histone deacetylase. The required change in the activity of the histone deacetylase may be an increase or a decrease in the activity of the histone deacetylase; a particular magnitude (for example, percentage) change in activity may be required in order for the compound to be identified. The change in histone deacetylase activity caused by a compound may be expressed as an IC₅₀, as well known to those skilled in the art ie the concentration of compound required to reduce the activity to 50% of its level in the absence of the compound. A particular IC₅₀ may be stipulated in order for the compound to be identified (ie the required change in activity may be expressed in terms of an IC₅₀).

Suitable methods of measuring or detecting the binding of the compound to the histone deacetylase or binding of the histone deacetylase to activated glucocorticoid receptor (GR) will be apparent to those skilled in the art. For example, a surface plasmon resonance assay, for example similar to that described in Plant et al (1995) Analyt Biochem 226(2), 342-348, may be used. Methods may make use of a polypeptide (or compound) that is labelled, for example with a radioactive or fluorescent label.

The method may be capable of high throughput operation, for example a chip-based method, for example in which the compounds to be tested are immobilised in a microarray on a solid support, as known to those skilled in the art.

Further examples may include cell based assays and protein-protein binding assays. An SPA-based (Scintillation Proximity Assay; Amersham International) system may be used. Conveniently this is done in a 96-well format. Other methods of detecting polypeptide/polypeptide interactions include ultrafiltration with ion spray mass spectroscopy/HPLC methods or other physical and analytical methods. Fluorescence Energy Resonance Transfer (FRET) methods, for example, well known to those skilled in the art, may be used, in which binding of two fluorescent labelled entities may be measured by measuring the interaction of the fluorescent labels when in close proximity to each other.

The yeast two-hybrid system may be used, as well known to those skilled in the art, where the histone deacetylase can be used to “capture” activated glucocorticoid receptor (GR). The yeast two-hybrid system is described in Fields & Song,

Nature 340:245-246 (1989).

It will be understood that it will be desirable to identify compounds that may modulate the activities of the polypeptide or polypeptides in vivo. Thus it will be understood that reagents and conditions used in the method may be chosen such that the interactions between the interacting polypeptides (histone deacetylase (and/or accessory proteins) and glucocorticoid receptor (GR)) are substantially the same as between the naturally occurring interacting polypeptides in vivo.

A second aspect of the invention provides a screening method for identifying a drug-like compound or lead compound for the development of a drug-like compound wherein the ability of a xanthine or related compound to modulate the expression of a histone deacetylase gene, or expression from a transcriptional regulatory sequence (for example, a promoter sequence) derived from a histone deacetylase gene, is measured and any compound capable of effecting the required modulation in the expression of the said histone deacetylase gene, or in the expression from the said transcriptional regulatory sequence, is identified. Techniques suitable for performing such a method will be known to those skilled in the art and are described in Example 1 and 2 and in the Figure legends (see, for example, the legend to FIG. 8). Preferably, the method comprises the steps of (1) exposing a cell to a xanthine or related compound, (2) measuring the change in expression of histone deacetylase or in expression from a transcriptional regulatory sequence derived from a histone deacetylase gene and (3) identifying any compound capable of effecting the required modulation in the expression of the said histone deacetylase or expression from the said transcriptional regulatory sequence.

The intention of the screen is to identify compounds that are capable of modulating the expression of a histone deacetylase from a histone deacetylase gene (ie a wild-type histone deacetylase gene) in a cell. The expression of the histone deacetylase, may be increased or decreased; preferably it is increased. The change in expression level of the histone deacetylase may be measured, for example, by determining the change in histone deacetylase activity; by determination of the amount of histone deacetylase polypeptide, for example using immunoassay techniques such as Western blotting, for example as described in Examples 1 and 2 and as discussed further below; or by determination of the amount of mRNA (or derived cDNA) encoding the histone deacetylase, for example using well known techniques including PCR-based techniques, for example as used in Example 1. It is preferred that the change in expression of histone deacetylase 1, 2 and/or 3 is measured, as discussed further below.

As well known to those skilled in the art, expression from a transcriptional regulatory sequence from a histone deacetylase gene may be measured by measuring expression of histone deacetylase from the gene comprising the transcriptional regulatory sequence. Alternatively, expression from a recombinant construct comprising the transcriptional regulatory sequence and a sequence (under the transcriptional control of the said regulatory sequence) encoding a “reporter” protein may be measured, as well known to those skilled in the art. A reporter protein may be one whose activity may easily be assayed, for example β-galactosidase, chloramphenicol acetyltransferase or luciferase (see, for example, Tan et al (1996)). In a further example, the reporter gene may be fatal to the cells, or alternatively may allow cells to survive under otherwise fatal conditions. Cell survival can then be measured, for example using calorimetric assays for mitochondrial activity, such as reduction of WST-1 (Boehringer). WST-1 is a formosan dye that undergoes a change in absorbance on receiving electrons via succinate dehydrogenase.

The cell may be an epithelial or inflammatory cell or cell line, examples of which are indicated above and in Examples 1 and 2. Preferably, the cell is an A549 cell, as described in Examples 1 and 2.

The screens may be used for identifying compounds which may be useful as a drug-like compound or lead compound for the development of a drug-like compound for treating (for example) abnormal cellular proliferation or differentiation; or, more preferably, inflammation, particularly asthma or other inflammatory airway disease, for example COPD (chronic obstructive pulmonary disease).

Processes for the production of xanthines are well known to those skilled in the art and are also described, for example, in EP 0 011 609, Belgian patent No 602888 and EP 0 089 028, all incorporated herein by reference. The screens of the invention may be performed using test compounds which may form part of a library of xanthines or related compounds. Such a library may be formed by techniques of combinatorial chemistry, as known to those skilled in the art. WO97/35990, for example (incorporated herein by reference), describes and provides references concerning techniques useful in preparing and screening a library of compounds, discussed further below.

By “related compound” is meant a compound, at least part of which may adopt a conformation substantially similar to those parts of a xanthine, for example theophylline, that appear, for example from a structure-activity relationship, to be important in modulating the activity of histone deacetylase. Such parts of a xanthine may interact with a histone deacetylase. This may be determined by molecular modelling, using techniques known to those skilled in the art. Such a compound may be able to bind to and/or modulate the activity of a histone deacetylase in a manner substantially similar to a xanthine, for example theophylline. The crystal structure of a histone deacetylase is reported in Finnin et al (1999) Nature 401(6749):188-93 “Structures of a histone deacetylase homologue bound to the TSA and SAHA inhibitors.”. The crystal structures are available, for example through the MEDLINE™ database, as records 11161(IC3R); 11162 (IC3S) and 11160 (IC3P).

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons molecular weight. A drug-like compound may additionally exhibit features of selective interaction with a particular protein or proteins and be bioavailable and/or able to penetrate cellular membranes, but it will be appreciated that these features are not essential. The drug-like compound may, however, be a compound useful as (and can be considered to be) a drug.

The term “lead compound” is similarly well known to those skilled in the art, and may include the meaning that the compound, whilst not itself suitable for use as a drug (for example because it is only weakly potent against its intended target, non-selective in its action, unstable, difficult to synthesise or has poor bioavailability) may provide a starting-point for the design of other compounds that may have more desirable characteristics.

It is preferred that the compound is a xanthine, preferably a methylxantine.

The uses indicated below (for example in the fourth and fifth aspects of the invention) or methods may be performed in vitro, either in intact cells or tissues, with broken cell or tissue preparations or at least partially purified components. Alternatively, they may be performed in vivo. Preferred uses or methods are described in the Examples. A particularly preferred screening method is described in Example 3. The cells tissues or organisms in/on which the use or methods are performed may be transgenic. In particular they may be transgenic for a particular histone deacetlyase under consideration or for a further histone deacetylase.

As noted above, it is preferred that the assay is capable of being performed in a “high throughput” format. This may require substantial automation of the assay and minimisation of the quantity of a particular reagent or reagents required for each individual assay. A scintillation proximity assay (SPA) based system, as known to those skilled in the art, may be beneficial.

It is preferred that the histone deacetylase activity is prepared from a total cellular homogenate, as described in Example 2 and Kolle et al (1998) “Biochemical methods for anlaysis of histone deacetylases” Methods 15, 323-331. It is further preferred that the histone deacetylase activity is provided as a crude preparation or immunoprecipitate, as described in Kolle et al (1998) and Example 2, ie that the xanthine or related compound is exposed to such a crude histone deacetylase preparation (or immunoprecipitate). It is further preferred that the preparation is obtained from epithelial or inflammatory cells or cell lines, for example macrophages or macrophage-like cultured cells.

It is preferred that the histone deacetylase activity comprises histone deacetylase 1, histone deacetylase 2 and/or histone deacetylase 3, preferably the human said deacetylase, still more preferably histone deacetylase 1. Methods for determining the presence (or expression levels) of each such histone deacetylase are well known to those skilled in the art and are described in Example 2. For example, human histone deacetylases 1 and 2 (HDAC1 and HDAC2) may be detected using rabbit polyclonal antibodies directed against HDAC1 or HDAC2, available from Santa-Cruz Biotechnology, Santa Cruz, Calif. Human deacetylase 3 may similarly be detected using a rabbit or goat polyclonal antibody available from Santa-Cruz Biotechnology.

It will be appreciated that compounds may be tested for activity against individual (for example, purified recombinant) histone deacetylases, and compounds which have different effects on different histone deacetylases (or different effects on the expression of different histone deacetylases) may be selected. For example, a compound may be selected which is specific for a histone deacetylase expressed in a particular cell or tissue type.

It is preferred that the compound increases the histone deacetylase activity and/or increases the binding of the histone deacetylase to the activated glucocorticoid receptor. Methods for measuring histone deacetylase activity are well known to those skilled in the art and are described in the Examples and in WO97/35990, incorporated herein by reference. Methods pertaining to measuring the binding of the histone deacetylase to, the activated glucocorticoid receptor are described, for example, in Examples 1 and 2. Methods of detecting binding of a compound to a polypeptide, for example the histone deacetylase, are well known to those skilled in the art. Examples of suitable methods are indicated in WO97/35990. The binding constant for the binding of the compound to the polypeptide may be determined. Suitable methods for detecting and/or measuring (quantifying) the binding of a compound to a polypeptide are well known to those skilled in the art and may be performed, for example, using a method capable of high throughput operation, for example a chip-based method. New technology, called VLSIPS™, for example, has enabled the production of extremely small chips that contain hundreds of thousands or more of different molecular probes. See, for example U.S. Pat. No. 5,874,219 issued Feb. 23, 1999 to Rava et al. These biological chips or arrays have probes arranged in arrays, each probe assigned a specific location. Biological chips have been produced in which each location has a scale of, for example, ten microns. The chips can be used to determine whether target molecules interact with any of the probes on the chip. After exposing the array to target molecules under selected test conditions, scanning devices can examine each location in the array and determine whether a target molecule has interacted with the probe at that location.

The methods, particularly methods wherein the ability of the histone deacetylase to bind to an activated glucocorticoid receptor is measured, may be performed in the presence of a glucocorticoid. The term glucocorticoid is well known to those skilled in the art. Suitable glucocorticoids include those routinely used in the treatment of inflammation, for example in the treatment of asthma. These are discussed in, for example, Martindale, The Extra Pharmacopoeia, 32^{nd} edition. Examples include dexamethasone and beclamethasone.

It will be appreciated that it is preferred that the compound acts directly on the histone deacetylase, but that the compound may act indirectly on the histone deacetylase. The compound may activate the histone deacetylase by modulating its phosphorylation state, as discussed in Example 2.

Lead compounds identified by the screening method of the invention may be developed further, for example by molecular modelling/and or experiments to determine the structure activity relationship, for example for modulators of a particular histone deacetylase, in order to develop more efficacious compounds, for example by improving potency, selectivity/specificity and pharmacokinetic properties.

The screening method of the first or second aspect of the invention may thus further comprise the steps of (1) exposing the compound to a phosphodiesterase activity and determining the effect of the compound on the phosphodiesterase activity and/or (2) exposing the compound to an adenosine receptor and determining the activity of the compound as an adenosine receptor antagonist and (3) any compound capable of the required effect on phosphodiesterase activity and/or having the required activity as an adenosine receptor antagonist is identified. Methods of carrying out these additional steps are well known to those skilled in the art and are discussed, for example, in the Examples and references contained therein.

It will be appreciated that improvements in pharmacokinetic properties may be particularly desirable with respect to theophylline and related compounds in view of the poor pharmacokinetic profile of theophylline. Compounds with no or reduced (when compared with theophylline) adenosine receptor antagonistic activity and/or no or reduced phosphodiesterase activity (when compared with theophylline) are preferably identified and selected. Such compounds may have reduced undesirable side effects when compared with, for example, theophylline. Thus, the required adenosine receptor antagonist activity may be that of theophylline or lower. The required phosphodiesterase inhibitory activity may be that of theophylline or lower.

It may further be desirable to determine whether the compound is metabolised by a cytochrome P450, using methods well known in the art. It is preferred that the compound is not metabolised by a cytochrome P450 as this may reduce interactions with other drugs. Nevertheless, the invention envisages that compounds identified in the screening methods of the invention as drugs or drug-like compounds may usefully be used as the basis for preparing prodrugs which, when administered to the patient, are converted to the active drug. This conversion may be carried out by, for example, a cytochrome P450.

A third aspect of the invention is a compound identifiable or identified by the screening methods of the first and second aspects of the invention, wherein the compound is not theophylline, caffeine, acepifylline, bamifylline, bufylline, cafaminol, cafedrine, diprophylline, doxofylline, enprofylline, etamiphylline, etofylline, proxyphylline, suxamidofylline, theobromine or a salt thereof. Thus, the third aspect of the invention includes histone deactetylase-activity-modulating xanthines or related compounds provided that these are not the compounds listed as excluded.

A fourth aspect of the invention provides a method for modulating a histone deacetylase activity wherein the histone deacetylase is exposed to a compound identifiable or identified by the screening method of the first or second aspects of the invention.

A fifth aspect of the invention provides the use of a compound identifiable or identified by the screening method of the first or second aspects of the invention in a method for modulating a histone deacetylase activity wherein the histone deacetylase is exposed to a compound identifiable or identified by the first or second aspects of the screening method of the invention.

It is preferred that the xanthine is a methylxanthine ie a methylated xanthine, preferably theophylline, theobromine or caffeine or any salt thereof. Alternatively, the xanthine may be acepifylline, bamifylline, bufylline, cafaminol, cafedrine, diprophylline, doxofylline, enprofylline, etamiphylline, etofylline, proxyphylline, suxamidofylline, theobromine or a salt thereof. It is preferred that the xanthine is an anti-asthmatically effective xanthine, for example as discussed in GB 2 163 957. The structures of suitable compounds are indicated in FIG. 19. Processes for the production of xantliines are well known to those skilled in the art and are also described, for example, in EP 0 011 609, Belgian patent No 602888 and EP 0 089 028, all incorporated herein by reference.

As well known to the skilled person, salts which may be conveniently used in therapy (and in screening methods) include physiologically acceptable base salts, for example, derived from an appropriate base, such as an alkali metal (eg sodium), awline earth metal (eg magnesium) salts, ammonium and NX₄⁺ (wherein X is C₁₋₄ alkyl) salts. Physiologically acceptable acid salts include hydrochloride, sulphate, mesylate, besylate, phosphate and glutamate. Salts may be prepared in conventional manner, for example by reaction of the parent compound with an appropriate base to form the corresponding base salt, or with an appropriate acid to form the corresponding acid salt. Examples of salts of theophylline, for example, are given in GB 2 163 957, incorporated herein by reference.

A still further aspect of the invention provides a compound as defined in the third aspect of the invention for use in medicine.

It will be appreciated that such a compound may be an inhibitor or activator of the histone deacetylase activity used in the screen and that the intention of the screen is to identify compounds that act as inhibitors or activators of the histone deacetylase, even if the screen makes use of a binding assay rather than an enzymic activity assay. It will be appreciated that the inhibitory/stimulatory action of a compound found to bind the histone deacetylase may be confirmed by-performing an assay of enzymic activity in the presence of the compound.

The purpose of the screen is to identify compounds useful in treating conditions caused by or exhibiting abnormal cellular proliferation or differentiation, for example cancer; or, more preferably, inflammation, particularly asthma or other inflammatory airway disease, for example COPD.

It will be appreciated that a recombinant histone deacetylase may be used in a method or use of the invention. The polynucleotide encoding the histone deacetylase may be mutated in order to encode a variant of the histone deacetylase, for example by insertion, deletion, substitution, truncation or fusion, as known to those skilled in the art. It is preferred that the histone deacetylase is not mutated in a way that may materially affect its biological behaviour, for example its enzymatic activity ie its histone deacetylase enzymic activity. References for nucleotide sequences encoding histone deacetylases are given, for example, in the database records referred to above.

It will be appreciated that in the discussion which follows of diseases and conditions in which xanthines useful as modulators, particularly activators, of histone deacetylase, for example theophylline may be useful, forms of conditions or diseases understood to be caused by excess phosphodiesterase activity or excessive adenosine receptor activity, or other target of xanthine, for example theophylline, action, for which xanthines have previously been suggested to be useful, are excluded. It is preferred that the forms of the conditions or diseases in which xanthines may be useful are forms in which histone deacetylase or the level of histone acetylation may be implicated or involved in their cause or exacerbation.

A still further aspect of the invention is the use of a compound identifiable by the screening method of the first or second aspects of the invention in the manufacture of a medicament for the treatment of a patient in need of modulation of histone deacetylase activity, wherein the patient is not in need of modulation of histone deacetylase activity on account of having asthma (or other inflammatory airway disease, for example COPD).

Preferably the compound is a compound according to the third aspect of the invention.

The patient may be a patient with anomalous cell proliferation, for example cancer, for example leukaemia, or fibroproliferative disorders. Alternatively, the patient may be a patient with anomalous cell differentiation, for example a neurodegenerative disease or disorders associated with connective tissue.

A further aspect of the invention provides the use of a compound identified or identifiable by a screening method of the first or second aspects of the invention in which a compound which increases histone deacetylation activity or increases binding to the activated glucocorticoid receptor is selected, in the manufacture of a medicament for the treatment of a patient in need of an increase in histone deacetylase activity or a decrease in histone acetylation, wherein the patient is not in need of modulation of histone deacetylase activity on account of having asthma (or other inflammatory airway disease, for example COPD).

An activator of histone deacetylase may be useful in causing differentiation, for example of hematopoietic cells, neuronal cells or other stem/progenitor cell populations, or for inducing apoptosis or other forms of cell death.

A further aspect of the invention provides the use of a compound of the third aspect of the invention in the manufacture of a medicament for the treatment of a patient with asthma (for example severe asthma) or other (preferably inflammatory) airway disease, for example chronic obstructive pulmonary disease (COPD), or other chronic inflammatory disease, including ulcerative colitis, rheumatoid arthritis and psoriasis.

Severe asthma includes asthma in which steroids alone are clinically not effective at doese that do not produce undesirable side-effects.

A further aspect of the invention provides the use of a compound identified or identifiable by a screening method of the first or second aspects of the invention in the manufacture of a medicament for the treatment of a disorder of cellular differentiation and/or proliferation in which excessive phosphodiesterase 3 or 4 activity or excessive adenosine receptor activity have not been implicated, but in which histone deacetylase or the level of histone acetylation has been implicated in causing or exacerbating the disorder. The disorder is not asthma (or other inflammatory airway disease, for example COPD). Examples of such disorders may include other chronic inflammatory diseases including ulcerative colitis, rheumatoid arthritis and psoriasis.

A further aspect of the invention provides a method of treatment of a patient in need of modulation of histone deacetylase activity, comprising administering an effective amount of a compound identified or identifiable by a screening method of the first or second aspects of the invention, wherein the patient is not in need of modulation of histone deacetylase activity on account of having asthma (or other inflammatory airway disease, for example COPD).

A further aspect of the invention provides a method of treatment of a patient in need of an increase in histone deacetylase activity or a decrease in histone acetylation, comprising administering an effective amount of a compound identifiable by a screening method of the first or second aspects of the invention in which a compound which increases histone deacetylation expression or activity or binding to the activated glucocorticoid receptor is selected, wherein the patient is not in need of modulation of histone deacetylase activity on account of having asthma (or other inflammatory airway disease, for example COPD).

A further aspect of the invention provides a method of treatment of a patient with asthma or other (preferably inflammatory) airway disease, for example COPD, comprising administering an effective amount of a compound of the third aspect of the invention.

A further aspect of the invention provides a method of treatment of a patient in need of modulation of histone deacetylase or histone acetylation, or with a disorder of cellular differentiation and/or proliferation in which excessive phosphodiesterase 3 or 4 activity or excessive adenosine receptor activity have not been implicated, but in which histone deacetylase or the level of histone acetylation has been implicated in causing or exacerbating the disorder, comprising administering an effective amount of a compound identifiable by a screening method of the first or second aspects of the invention. The disorder is not asthma (or other inflammatory airway disease, for example COPD). Examples of such disorders may include other chronic inflammatory diseases including ulcerative colitis, rheumatoid arthritis and psoriasis.

In any of the above treatment-related aspects of the invention, it is preferred that a glucocorticoid is, has been, or will be administered to the patient, in addition to the indicated compound. Suitable glucocorticoids will be known to those skilled in the art and may include dexamethasone and/or beclamethasone, as discussed above. Co-administration of a steroid with the indicated compound may be particularly beneficial for patients with severe asthma or COPD, as discussed in the Examples.

A further aspect of the invention provides a kit of parts comprising a glucocorticoid and a compound of the third aspect of the invention. A further aspect of the invention provides a composition comprising a glucocorticoid and a compound of the third aspect of the invention. Preferably, the composition is a pharmaceutical composition and includes a pharmaceuticlaly acceptable carrier.

A still further aspect of the invention provides a kit of parts suitable for carrying out a screening method of the invention comprising a histone deacetylase and a xanthine or related compound, as defined above.

The kit of parts may further comprise a glucocorticoid.

As will be clear from the above, the invention provides the use of a histone deacetylase in a method of identifying a compound useful for treating asthma (or other inflammatory airway disease), ulcerative colitis and/or rheumatoid arthritis.

It further provides a screening method for identifying a drug-like compound or lead compound for the development of a drug-like compound for treating asthma (or other inflammatory airway disease), ulcerative colitis and/or rheumatoid arthritis, in which (1) a test compound is exposed to a histone deacetylase, (2) the binding of the compound to the histone deacetylase is measured or the change in the activity of the histone deacetylase is measured or the change in the ability of the histone deacetylase to bind to activated glucocorticoid receptor (GR) is measured and (3) any compound capable of the required binding to the histone deacetylase or producing the required change in the activity of the histone deacetylase or its ability to bind to activated glucocorticoid receptor is identified.

It further provides a screening method for identifying a drug-like compound or lead compound for the development of a drug-like compound for treating asthma (or other inflammatory airway disease), ulcerative colitis and/or rheumatoid arthritis, wherein the ability of a test compound to modulate the expression of a histone deacetylase gene, or expression from a transcriptional regulatory sequence (for example, a promoter sequence) derived from a histone deacetylase gene, is measured and any compound capable of effecting the required modulation in the expression of the said histone deacetylase gene, or in the expression from the said transcriptional regulatory sequence, is identified.

In the preceding two aspects of the invention, the test compound is not limited to being a xanthine or xanthine-related compound.

A further aspect of the invention provides the use of a compound which increases histone deacetylase activity in the manufacture of a medicament for treatment of a patient with asthma or other inflammatory airway disease (for example COPD), ulcerative colitis or rheumatoid arthritis wherein the compound is not theophylline, caffeine, acepifylline, bamifylline, bufylline, cafaminol, cafedrine, diprophylline, doxofylline, enprofylline, etamiphylline, etofylline, proxyphylline, suxamidofylline, theobromine or a salt thereof, or a glucocorticoid or pyridinylimidazole compound.

A further aspect of the invention provides a method of treatment of a patient with asthma or other inflammatory airway disease (for example COPD) comprising administering an effective amount of a compound which increases histone deacetylase activity, wherein the compound is not theophylline, caffeine, acepifylline, bamifylline, bufylline, cafaminol, cafedrine, diprophylline, doxofylline, enprofylline, etamiphylline, etofylline, proxyphylline, suxamidofylline, theobromine or a salt thereof, or a glucocorticoid or pyridinylimidazole compound.

The patient may also be administered a corticosteroid, as discussed above. A further aspect of the invention provides a kit of parts comprising a said compound and a corticosteroid. A still further aspect of the invention provides a composition comprising a said compound and a corticosteroid. Preferably, the composition is a pharmaceutical composition which includes a pharmaceutically acceptable carrier.

The compound which increases histone deacetylase activity may be a recombinant polynucleotide expressing a histone deacetylase or other stimulator of histone deacetylase activity, for example as described in WO97/35990.

The administered compounds (for example, the compounds of the third aspect of the invention or compounds identified or identifiable by the sceening methods of the invention, or said compound which increase histone deacetylase activity) may be administered in any suitable way, usually parenterally, for example intravenously, intraperitoneally or intravesically, in standard sterile, non-pyrogenic formulations of diluents and carriers. The compounds of the invention may also be administered topically, for example to the lungs, for example using an inhaler system as well known to those skilled in the art. The compounds of the invention may also be administered in a localised manner, for example by injection.

It will be appreciated that a further aspect of the invention provides a composition comprising a compound of the third aspect of the invention and a pharmaceutically acceptable excipient.

The invention is now described in more detail by reference to the following, non-limiting, Figures and Examples:

### FIGURE LEGENDS

FIG. 1. Histone acetylation is associated with IL-1β-and dexamethasone-induced gene expression.

(A) Time course of IL-1β-induced GM-CSF release. Cells were incubated with IL-1β (1 ng/ml) for the times indicated and GM-CSF released into the medium measured by ELISA. Results are expressed as mean±SEM, n=at least 3 independent experiments.

(B) Effect of Trichostatin A (TSA) on histone deacetylase activity and histone H4 acetylation. Cells were treated with increasing concentrations of TSA for 6 hrs before total cellular proteins were isolated and analysed for deacetylase activity and also histone acetylation by Western blotting using an anti-pan acetylated histone H4 antibody. Results are expressed as mean±SEM and are representative of at least 3 independent experiments, **p<0.01.

(C) Inhibitory effects of dexamethasone on IL-1β-induced GM-CSF and SLPI production. Cells were preincubated with various concentrations of dexamethasone for 1 hr before incubation with IL-1β (1 ng/ml) for 6 hours. Supernatants were collected and assayed for GM-CSF and SLPI by ELISA. The effects of TSA (1 ng/ml) on IL-1β-stimulated GM-CSF and SLPI release were also measured. Results are expressed as mean±SEM, n=at least 3 independent experiments, **p<0.01.

(D) Time and concentration-dependent histone H4 acetylation by IL-1β. Western blot analysis of time—(left) and concentration—(right) dependent histone acetylation by IL-1β. Cells were either incubated with IL-1β (1 ng/ml) for the time indicated or incubated with different concentrations of IL-1β for 6 hr. The result is representative of 3 independent experiments. [³H-acetate incorporation assay for time—(left) and concentration—(right) dependent histone acetylation by IL-1β. Data represents mean±SEM of 3 independent experiments. *p<0.05, **p<0.01.

(E) Time and concentration-dependent histone H4 acetylation by dexamethasone. Western blot analysis of time—(left) and concentration—(right) dependent histone acetylation by dexamethasone. Cells were incubated with either dexamethasone (10⁻⁶ M) as indicated or with different concentrations of dexamethasone for 6 hr. Result is representative of 3 independent experiments. [³H]-acetate incorporation assay for time—(left) and concentration—(right) dependent histone acetylation by dexamethasone was also performed. Data represents mean±SEM of 3 independent experiments. *p<0.05, **p<0.01.

FIG. 2. IL-1β and dexamethasone acetylate specific and distinct lysine residues.

Immunocytochemical staining for specific histone H4 acetylated lysine residues. Cells were incubated with dexamethasone (10⁻⁷M)(b, f, j & n), IL-1β (1 ng/ml)(c, g, k & o) or TSA (100 ng/ml)(d, h, l & p) for 6 hr (a-d) before probing with antibodies against the acetylated forms of histone H4 lysine residues K5 (a-d), K8 (e-h), K12 (I-l) and K16 (m-p). Results are representative of 4 independent experiments.

FIG. 3. Effects of dexamethasone on IL-1β-induced histone acetylation.

(A) Specific lysine acetylation by PCAF. Cells were treated with IL-1β (1 ng/ml) for 6 hrs before total cellular proteins were extracted. PCAF was immunoprecipitated under stringent IP conditions (see methods) and associated acetylated lysine residues detected by ELISA. Histone acetylation at each lysine residue is expressed in units (1 unit is equivalent to the absorbance produced by 50 ng of TSA-treated hyperacetylated histone). Results are expressed as mean±SEM, n=at least 3 independent experiments.

(B) Specific lysine acetylation by CBP. Cells were treated with IL-1β (1 ng/ml) for 6 hrs before total cellular proteins were extracted. CBP was immunoprecipitated under stringent IP conditions (see methods) and associated acetylated lysine residues detected by ELISA. Histone acetylation at each lysine residue is expressed in units (1 unit is equivalent to the absorbance produced by 50 ng of TSA-treated hyperacetylated histone). Results are expressed as mean±SEM, n=at least 3 independent experiments.

(C) Specific lysine acetylation by CBP. Cells were treated with IL-1β (1 ng/ml) for 6 hrs before total cellular proteins were extracted. CBP was immunoprecipitated under mild IP conditions (see methods) and associated acetylated lysine residues detected by ELISA. Histone acetylation at each lysine residue is expressed in units (1 unit is equivalent to the absorbance produced by 50 ng of TSA-treated hyperacetylated histone). Results are expressed as mean±SEM, n=at least 3 independent experiments.

(D) Histone acetylation by IL-1β in the presence of dexamethasone in whole cell extracts. Cells were pretreated with dexamethasone for 1 hr before incubation with IL-1β (1 ng/ml) for 6 hr in the presence of 0.05 mCi [³H]-acetate. Histones were isolated and separated by SDS-PAGE and [³H]-acetate incorporated histones were counted and normalised to protein level. The effect of dexamethasone alone and of TSA on dexamethasone suppression of IL-1β-stimulated histone acetylation was also investigated. Data represents mean±SEM of 3 independent experiments. **p<0.01.

(E) Western blot analysis of dexamethasone actions on IL-1β-stimulated histone acetylation. Cells were incubated with IL-1β (1 ng/ml) for 6 hrs in the presence or absence of different concentrations of dexamethasone. Protein extracts were obtained and examined for pan acetylated histone H4 lysine residues and for specific K5, K8, K12 and K16 acetylation by Western blotting. Control (lane 1); IL-1β stimulation (lane 2); IL-1β stimulation in the presence of dexamethasone at 10⁻¹²M (lane 3), 10⁻¹⁰M (lane 4), 10⁻⁸M (lane 5) and 10⁻⁶M (lane 6); dexamethasone, 10⁻⁶M alone (lane 7). Results are representative of 3 independent experiments.

FIG. 4. Association of specific acetylated lysine residues with GM-CSF and SLPI gene promoters.

(A) GM-CSF and SLPI promoter regions. The sequence of the GM-CSF (−191-−10) and SLPI (−170-+32) promoter regions amplified by PCR primer pairs. Primers are indicated by overlined sequences. The NF-κB response element in the GM-CSF promoter underlined. The coding region (CR) of each gene is indicated by an arrow. An enrichment of the GM-CSF promoter DNA is shown following PCR amplification of immunoprecipitation of p65 associated DNA from cells treated with IL-1β (1 ng/ml) for 1 hr.

(B) Specific lysine residue acetylation at the GM-CSF and SLPI promoters. Cells were incubated with IL-1β (1 ng/ml) in the presence or absence of various concentrations of dexamethasone. Proteins and DNA were cross-linked by formaldehyde treatment and chromatin pellets extracted. Following sonication, acetylated histone H4 lysine residues (AcK5, AcK8 and AcK12) were immunoprecipitated and the associated DNA amplified by PCR. Results are representative of 3 independent experiments.

FIG. 5. Dexamethasone inhibits p65-associated histone acetylation: a role for HDAC.

(A) Dexamethasone inhibits IL-1β-induced p65 immunoprecipitated histone acetylation. Cells were preincubated with various concentrations of dexamethasone for 1 hr before IL-1β (1 ng/ml) treatment for a further 6 hrs. Total cellular proteins were isolated and p65 associated proteins immunoprecipitated under stringent conditions (see methods). The associated histone acetylation activity was measured following incubation of the p65-IP extract with 10 μg free core histones and 0.25 mCi of ³H-acetyl CoA for 45 minutes. Radiolabelled histones were counted and results presented as mean±sem of at least 3 independent experiments. **p<0.01.

(B) Effect of TSA on dexamethasone inhibition of p65-associated histone acetylation. Histone acetylation experiments were performed as in (A) in the presence of TSA (100 ng/ml). This produced a reduced ability of dexamethasone to suppress p65-associated histone acetylation. Results are presented as mean±sem of at least 3 independent experiments. **p<0.01.

(C) Effect of Il-1β and dexamethasone on p65-associated histone deacetylation. Using the same immunoprecipitates as in (A) histone deacetylase activity was measured by incubation of extracts with ³H-labelled histones for 30 mins. Free ³H-labelled acetic acid was extracted by ethylacetate and measured by liquid scintillation counting. Results are presented as mean±sem of at least 3 independent experiments. **p<0.01.

(D) Specific lysine acetylation by p65. Cells were treated with IL-1β(1 ng/ml) for 6 hrs before total cellular proteins were extracted. p65 was immunoprecipitated under stringent IP conditions (see methods) and associated acetylated lysine residues detected by ELISA. Histone acetylation at each lysine residue is expressed in units (1 unit is equivalent to the absorbance produced by 50 ng of TSA-treated hyperacetylated histone). Results are expressed as mean±SEM, n=at least 3 independent experiments.

FIG. 6. Effect of dexamethasone on p65-associated co-activators and GR recruitment.

(A) Effect of dexamethasone on CBP and PCAF expression. Cells were incubated with vehicle (control), dexamethasone 10⁻⁸ M (lane 2) and 10⁻⁶ M (lane 3) for 6 hr. Proteins were extracted and size fractionated by SDS-PAGE and CBP and PCAF detected by Western blotting. Results are representative of 3 independent experiments.

(B) Effect of dexamethasone on CBP/p65 interaction and PCAF/p65 interaction. Cells were preincubated with vehicle (lane 1), IL-1β (1 ng/ml) (lane 2) or IL-1β and dexamethasone (10⁻⁶ M) for 1 hr (lane 3) before total cellular proteins were extracted. Immunoprecipitation was performed with anti-p65 antibody in mild IP buffer (see methods). Immunoprecipitates were separated by SDS-PAGE and detected by Western blotting using anti-CBP or PCAF antibody. The bottom panel shows p65 presence in nuclear extracts. Results are representative of 3 independent experiments.

(C) Effect of dexamethasone on CBP/PCAF interaction. Cells were preincubated with vehicle (lane 1), IL-1β (1 ng/ml) (lane 2) or IL-1β and dexamethasone (10⁻⁶M) (lane 3) before protein extraction and PCAF immunoprecipitation under mild IP conditions. Results are representative of 3 independent experiments.

(D) Effect of dexamethasone on CBP phosphorylation. Cells were incubated with [³²P] orthophosphate for 30 min before stimulation with IL-1β (1 ng/ml) for 6 hr in the absence (lane 2) or presence (lane 3) of dexamethasone (10⁻⁶M). Cells were collected, total cellular proteins extracted and immunoprecipitated with anti-CBP antibody. Results were visualised by autoradiography (upper panel) and compared to immunoprecipitated CBP as measured by Western blotting (lower panel). Unstimulated cells (lane 1) and blocking peptide (lane 4) were used as controls. These results are representative of 3 independent experiments. The results from these and other experiments where the radioactive bands were excised and counted are also shown in the right hand panel. Results are expressed as the mean±SEM of 3 independent experiments.

FIG. 7. Effect of dexamethasone on IL-1β-stimulated CBP-associated histone acetylation and deacetylation activity.

(A) Effect of IL-1β and dexamethasone on PCAF immunoprecipitated histone acetylation. Cells were preincubated with various concentrations of dexamethasone 1 hr before IL-1β (1 ng/ml) treatment for 6 hrs. Total cellular proteins were extracted and PCAF immunoprecipitated under stringent IP conditions (see methods). The associated histone acetylation activity was measured following incubation of the PCAF-IP extract with 10 μg free core histones and 0.25 mCi of ³H-acetyl CoA for 45 minutes. Alkali precipitated radiolabelled histones were counted and results presented as mean±sem of at least 3 independent experiments.

(B) Effect of dexamethasone on IL-1β-stimulated CBP immunoprecipitated histone acetylation. Cells were treated as in (A) and CBP immunoprecipitated under stringent conditions (see methods). Histone acetylation was measured as in (A) and results presented as mean±sem of at least 3 independent experiments, *p<0.05.

(C) Effect of dexamethasone on IL-1β-stimulated CBP-associated histone acetylation. Cells were treated as in (A) and CBP immunoprecipitated under mild conditions (see methods). Histone acetylation was measured as in (A). A blocking peptide to the CBP antibody completely blocked specific histone acetylation. Results presented as mean±sem of at least 3 independent experiments, *p<0.05.

(D) Dexamethasone suppression of IL-1β-induced CBP-associated HAT activity requires GR. Cells were treated with IL-1β (1 ng/ml) alone for 6 hrs, cellular proteins extracted and CBP immunoprecipitated under stringent conditions (see methods). Immunoprecipitated proteins were incubated with dexamethasone alone or a mixture of dexamethasone and highly purified GR together with ³H-acetyl CoA for 45 mins in the presence of TSA (100 ng/ml). The associated histone acetylation activity was measured as in (A) and results presented as mean±sem of at least 3 independent experiments, *p<0.05.

(E) Effect of IL-1β and dexamethasone on histone deacetylation. Using the same immunoprecipitates as in (C) above histone deacetylase activity was measured by incubation of extracts with ³H-labelled histones for 30 mins. Free ³H-labelled acetic acid was extracted by ethylacetate and measured by liquid scintillation counting. Results are presented as mean±sem of at least 3 independent experiments. *p<0.05, **p<0.01.

(F) Effect of IL-1β and dexamethasone on GR-mediated histone deacetylation. Cells were treated as in (A) and total cellular proteins were immunoprecipitated using an anti-GR antibody under stringent IP conditions (see methods). Histone deacetylase activity was measured by incubation of extracts with ³H-labelled histones for 30 mins. Free ³H-labelled acetic acid was extracted by ethylacetate and measured by liquid scintillation counting. Results are presented as mean±sem of at least 3 independent experiments.

FIG. 8. Effect of dexamethasone on HDAC protein expression, HDAC activity and HDAC recruitment to the p65 complex.

(A) Relative expression of HDAC1 and HDAC2 in A549 cells. Total cellular proteins from untreated A549 cells were isolated. 30 μg protein was size-fractionated by 10% SDS-PAGE and Western blot analysis performed using polyclonal anti-HDAC1 and HDAC2 antibodies. Results are representative of 3 independent observations.

(B) Effect of dexamethasone on HDAC2 protein expression. Cells were incubated with vehicle (control), dexamethasone 10⁻⁸ M (lane 2) and 10⁻⁶ M (lane 3) for 6 hr. Proteins were extracted and size fractionated by SDS-PAGE and HDAC2 detected by Western blotting. Densitometric analysis of HDAC2 expression is shown graphically in the lower panel. Data from 3 separate experiments was normalised to β-actin and results expressed as mean±SEM. *p<0.05.

(C) Effect of dexamethasone on histone deacetylation. Cells were incubated in the presence or absence of increasing concentrations of dexamethasone (10⁻¹⁰ M, 10⁻⁸ M, 10⁻⁶ M) for 6 hr. Total cellular proteins were isolated and histone deacetylation activity measured by incubation of extracts with ³H-labelled histones for 30 mins. Free ³H-labelled acetic acid was extracted by ethylacetate and measured by liquid scintillation counting. Results are expressed as mean±SEM of 3 separate experiments. *p<0.05.

(D) Recruitment of HDAC2 to p65 and GR immunoprecipitated complexes. Cells were incubated with IL-1β (1 ng/ml) in the presence or absence of dexamethasone (10⁻¹⁰ M) for 6 hr. Total cellular proteins were isolated and immunoprecipitated with anti-p65 or anti-GR antibodies using mild IP conditions (see methods). HDAC2 expression in the immunoprecipitated complexes was measured by Western blotting. p65 and GR expression in the same samples is shown as a control for protein loading. The result is representative of 3 separate experiments.

FIG. 9. Proposed model for dexamethasone/GR complex inhibition of IL-1β-stimulated histone acetylation

DNA bound p65 induces histone acetylation via activation of CBP and a CBP-associated HAT complex. This results in local unwinding of DNA and increased gene transcription. GR, possibly acting as a monomer, interacts with CBP causing an inhibition of CBP-associated HAT activity. In addition, GR also recruits HDAC2 to the activated p65/CBP complex further reducing local HAT activity leading to enhanced nucleosome compaction and repression of transcription.

FIG. 10. Effect of theophylline and dexamethasone (Dex) on histone acetylation and GM-CSF release in A549 cells. (a) Cells were stimulated with LPS (3 ng/ml) for 24 hours in the presence or absence of theophylline (10⁻⁵ M) or Dex (10⁻⁶ M). Total cellular proteins were extracted and histone acetylase activity measured. (b) GM-CSF release into the culture medium was measured after 6 hours by ELISA.

FIG. 11. Effect of theophylline on histone deacetylase (HDAC) activity in A549 cells. (a) Cells were stimulated with LPS (3 ng/ml) for 24 hours in the presence or absence of theophylline (10⁻⁵ M) or Dex (10⁻⁶ M). Total cellular proteins were extracted and histone deacetylase activity measured. (b) Direct effect of theophylline on HDAC activity. Nuclear proteins containing HDAC activity were isolated from untreated cells and incubated with [³H]-histones for 45 minutes in the presence of theophylline or dexamethasone. Results are expressed as mean±SEM (n=3-5, *p<0.05, **p<0.01).

FIG. 12. Effect of theophylline on HDAC expression. Western blot analysis was used to determine the effect of theophylline and dexamethasone on HDAC1 (upper panel) and HDAC2 (lower panel) expression in A549 cells after 24 hours. Band densities were controlled for protein loading by comparison with β-actin expression. Results are shown as relative band densities.

FIG. 13. Theophylline actions on HDAC activity do not occur through PDE4 inhibition or adenosine receptor antagonism. (a) Direct effect of the PDE4 inhibitor rolipram (10 μM) and the adenosine receptor antagonist CGS-15943 (10 μM) on HDAC activity. Nuclear proteins containing HDAC activity were isolated from untreated cells and incubated with [³H-histones for 45 minutes in the presence of theophylline, rolipram or CGS15943. (b) Direct effect of the MEK inhibitor PD089159 (1 μM) and the p38 MAPK inhibitor SB203580 on theophylline induced HDAC activity. Results are expressed as mean±SEM (n=3-5, *p<0.05, **p<0.01).

FIG. 14. Effect of theophylline on glucocorticoid actions in A549 cells.

(a) The effect of low concentration theophylline (T, 10⁻⁵ M) on dexamethasone (D) modulation of total cell HDAC activity. Cells were treated with T, D or T plus D for 6 hrs before nuclear proteins were isolated and HDAC activity measured. (b) IL-1β-stimulated GM-CSF release into the culture medium of IL-1β-stimulated cells in the presence of T, D or T plus D was determined by ELISA. Results are expressed as mean of 2 experiments.

FIG. 15. Effect of theophylline on HDAC expression and activity in vivo. HDAC1 and: HDAC2 localisation in bronchial biopsies from mild asthmatic patients.

FIG. 16. Effect of theophylline on HDAC expression and activity in vivo. (a) Western blot analysis of HDAC1 and HDAC 2 expression in bronchial biopsies from mild asthmatic subjects treated with low dose theophylline (LDT) or placebo. (b) Graphical expression of the effect of LDT and placebo on HDAC1 and HDAC2 expression relative to β-actin. (c) Effect of LDT and P on HDAC activity in bronchial biopsies. N=14.

FIG. 17. Effect of theophylline on HAT and HDAC activity in BAL macrophages. Macrophages were incubated for 24 hours in the presence of increasing concentrations of theophylline and dexamethasone. HAT activity (a) and HDAC activity (b) were measured as described in the methods. Results are expressed as mean±SEM (n=5).

FIG. 18. Structure of xanthine (dioxopurine; C₅H₄N₄O₂)

FIG. 19. Structures of anti-asthmatically effective xanthine compounds

FIG. 20. Effects of theophylline and dexamethasone on HDAC activity and IL-8 production in macrophages from non-smokers or smokers.

FIG. 21. Effect of theophylline on histone deacetylase activity and expression and cytokine production in IL-1β plus H₂O₂ stimulated A549 cells.

FIG. 22. Effect of theophylline on HDAC1, HDAC2 and HDAC3 activity.

FIG. 23. Effect of combination of low dose theophylline and low dose dexamethasone on HDAC activity and GM-CSF production in A549 cells.

### EXAMPLE 1

Glucocorticoid Receptor Recruitment of Histone Deacetylase 2 Inhibits IL-1β-Induced Histone H4 Acetylation on Lysines 8 and 12

We have investigated the ability of dexamethasone to regulate IL-1β-induced gene expression, histone acetyltransferase (HAT) and deacetylase (HDAC) activity. Low concentrations of dexamethasone (10⁻¹⁰M) repress IL-Iβ-stimulated granulocyte/macrophage-cell stimulating factor (GM-CSF) expression and fail to stimulate secretory leukocyte proteinase inhibitor (SLPI) expression. Dexamethasone (10⁻⁷M) and IL-1β (1 ng/ml) stimulated HAT activity but showed a different pattern of histone H4 acetylation. Dexamethasone targeted lysines K5 and K16, whereas IL-1β targeted K8 and K12. Low concentrations of dexamethasone (10⁻¹⁰M), which do not transactivate, repressed IL-1β-stimulated K8 and K12 acetylation. Using chromatin immunoprecipitation assays we show that dexamethasone inhibits IL-1β-enhanced K8-associated GM-CSF promoter association in a concentration dependent manner. Neither IL-1β nor dexamethasone elicited any GM-CSF promoter association at K5 acetylated residues. We show that the activated GR complex acts both as a direct inhibitor of CBP-associated HAT activity and also by recruiting HDAC2 to the p65/CBP HAT complex. This action does not involve de novo synthesis of HDAC protein or altered expression of CBP or p300/CBP associated factor (PCAF). This mechanism for glucocorticoid repression is novel and establishes that inhibition of histone acetylation is an additional level of control of inflammatory gene expression. This further suggests that pharmacological manipulation of specific histone acetylation status is a potentially useful approach for the treatment of inflammatory diseases.

Materials and Methods

Cell Culture

A549 cells were grown to 50% confluence in Dulbecco's modified medium (DMEM) containing 10% fetal calf serum (FCS) before incubation for 48-72 hr in serura-free media. Cells were stimulated by IL-1β (1 ng/ml) in the presence or absence of dexamethasone and the effects of the histone deacetylase inhibitor trichostatin A (TSA) (Sigma, Poole, UK) (Yoshida, M et al (1990) J. Biol. Chem. 265:17174-17179.) on baseline and IL-1β-stimulated expression of GM-CSF and SLPI release measured.

GM-CSF, SLPI and Acetylated Histone ELISAs

Determination of GM-CSF expression was measured by sandwich ELISA (Pharmingen, Lugano, Switzerland) according to the manufacturer's instructions. For immunoassay of SLPI and acetylated histone, polystyrene microtitre plates were coated overnight at 4° C. with sample diluted with hydroxy carbonate (pH 9.6). Plates were blocked for 2 hr with 5 % ovalbumin in PBS. Antibodies against SLPI (R&D Systems Europe, Abingdon, UK), K5, K8, K12 and K16 acetylated histone 4 (Serotec, Oxford, UK) were diluted 1:300-1:1000 and added to each plate. After 1 hr at room temperature plates were washed sequentially with 0.1% Tween20-PBS and incubated with HRP conjugated goat anti-rabbit antibody (DAKO, Cambridge, UK) for 1 hr. Detection was performed with ABTS following Pharmingen instructions. Recombinant human SLPI (R&D Systems Europe) was used as a standard. As a standard for acetylated histone, crude extracted histone from A549 cells incubated with TSA (100 ng/ml) for 6 hr was used, and the value was calculated in units, where 1 unit is equivalent to the absorbance of 50 ng of TSA-treated hyperacetylated histone after subtraction of BSA-induced histone acetylation.

Direct Histone Extraction

Histones were extracted from nuclei overnight using HCl and H₂SO₄ at 4° C. using a modified method from that as described by Turner (Turner, B. M. & G. Fellows (1989) Eur. J. Biochem 179:131-139; Yoshida, M et al (1995) Bioessays 17:423-430.). Cells were microfuged for 5 min and the cell pellets extracted with ice-cold lysis buffer (10 mM Tris-HCl, 50 mM sodium bisulphite, 1% Triton X-100, 10 mM MgCl₂, 8.6% sucrose, complete protease inhibitor cocktail (Boehringer-Mannheim, Lewes, UK) for 20 min at 4° C. The pellet was repeatedly washed in buffer until the supernatant was clear (centrifuge at 800 rpm, 5 min after each wash) and the nuclear pellet washed in nuclear wash buffer (10 mM Tris-HCl, 13 mM EDTA) and resuspended in 50 μl of 0.2 N HCl and 0.4 N H₂SO₄ in distilled water. The nuclei were extracted overnight at 4° C. and the residue microfuged for 10 min. The supernatant was mixed with 1 ml ice-cold acetone and left overnight at −20° C. The sample was microfuged for 10 min, washed with acetone, dried and diluted in distilled water. Protein concentrations of the histone containing supernatant were determined by Bradford protein assay kit (BioRad, Hemel Hempstead, UK).

Western Blotting

Immunoprecipitates, whole cell extractions or isolated histones were measured by SDS-PAGE and Western blot analysis using ECL (Amersham, Amersham, UK). Proteins were size-fractionated by SDS-PAGE and transferred to Hybond-ECL membranes. Immunoreactive bands were detected by ECL.

Immunocytochemistry

A549 cells (0.5×10⁶) were cultured in 8 well slide chambers with IL-1β (1 ng/ml) in the presence or absence of various concentrations of dexamethasone. Cells were washed with Hanks solution, and air-dried for 30 min at RT. Cells were then fixed in ice-cold acetone-methanol (50/50, w/w) (−20° C.) for 10 min. Slides were air dried and incubated with blocking buffer (20% normal swine serum in PBS, 0.1% saponin)(Dako) for 20 mmn followed by 1 hr incubation with primary antibody solution (PBS, 0.1% saponin, 1% BSA). Antibodies against pan-acetylated H4, H4-K5, H4-K8, H4-K12 and H4-K16 (Serotec) were used at 1:100 to 1:300 dilution. Slides were washed twice and incubated with biotinylated swine anti-rabbit IgG (Dako)(1:200) for 45 min. Slides were washed again before incubation with fluorescein isothiocyante-conjugated streptavidine (1:100) for 45 min. The slides were washed twice more before counterstaining with 20% haematoxyline, and mounting. Stained cells were observed by confocal microscopy. Confocal scanning laser microscopy images were collected with a Leica confocal microscope, equipped with a 488/514 nm dual band argon ion laser. An oil-immersion objective was used and images were collected using TCSNT software.

Histone Acetylation Activity

Cells were plated at a density of 0.25×10⁶ cells/ml and exposed to 0.05 mCi/ml of [³H] acetate (Amersham). After incubation for 10 min at 37° C. cells were stimulated for 6 hr. Histones were isolated and separated by electrophoresis on SDS-16% polyacrylamide gel. Gels were stained with Coommasie brilliant blue and the core histones (H2A, H2B, H3 and H4) excised. The radioactivity in extracted core histones was determined by liquid scintillation counting and normalised to protein level.

Histone Deacetylation Activity

Radiolabelled histones were prepared from A549 cells following incubation with TSA (100 ng/ml, 6 hr) in the presence of 0.1 mCi/ml [³H]-acetate. Histones were dried and resuspended in distilled water. Crude HDAC preparations were extracted from total cellular homogenates with Tris-based buffer (10 mM Tris-HCl pH 8.0, 500 mM NaCl, 0.25 mM EDTA, 10 mM 2-mercaptoethanol) as previously reported (Kolle, D et al (1998) Methods 15:323-331). The crude HDAC preparation or immunoprecipitates were incubated with [³H-labelled histone for 30 min at 30° C. before the reaction was stopped by the addition of 1N HCl/0.4N acetic acid. Released [³H]-labelled acetic acid was extracted by ethylacetate and the radioactivity of the supernatant was determined by liquid scintillation counting.

Immunoprecipitation

Extracts were prepared using 100 μl of stringent immunoprecipitation (IP) buffer (50 mM Tris-HCl, pH 8.0, 150 mM NaCl, 1.0% triton X-100, 0.5% NP-40, 0.1% SDS, 0.5% deoxycholate, complete protease inhibitor cocktail (Boehringer-Mannheim)) or mild IP buffer (10 mM Tris-HCl, pH 8.0, 150 mM NaCl, 0.5% NP-40, complete protease inhibitor cocktail (Boehringer-Mannheim)). The lysis mixture was incubated on ice for 15 min and microfuged for 10 min at 4° C. Extracts were precleared with 20 μl of A/G agarose (a 50:50 mix; Santa Cruz, Santa Cruz, Calif.) and 2 μg of normal IgG. After microcentrifugation, 20 μl of A/G agarose conjugated with 5 μg of antibody were used to precipitate CBP, PCAF, GR or p65 overnight at 4° C. with rotation. The immune complexes were pelleted by gentle centrifugation and washed 3 times with 1 ml of IP buffer. For the HAT assay, immunoprecipitates were washed twice with IP-HAT buffer, and for Western blotting, after final wash with IP buffer, the buffer was aspirated completely and resuspended in Laemmli buffer.

Purification of GR

GR was purified from 5×10⁹ A549 cells. Total cellular proteins were isolated and GR immunoprecipitated as above using a mouse anti-GR antibody (Serotec). The immunoprecipitate was separated by 8% SDS-PAGE and GR purified from the excised gel by electro-elution according to the manufacturer's instructions (Bio-Rad, Model 422) and used at a concentration of 1 ng/ml.

IP-HAT Assay

IP-HAT assays were performed using a modified method of Ogryzko (Ogryzko, V. V et al (1996) Cell 87:953-959). Immune complexes with resin were resuspended in 150 μl of HAT buffer (50 mM Tris-HCl, pH 8.0, 10% glycerol, 1 mM dithiothreitol, 0.1 mM EDTA, complete protease inhibitor cocktail). Typically, 20 μl of free core histone solution extracted from A549 cells (final amount 10 μg) and 30 μl of immunoprecipitate were incubated. Reactions were initiated by the addition of 0.25 mCi of [³H] acetyl-CoA (5 Ci/mmol)(Amersham) and performed for 45 min at 30° C. After incubation, the reaction mixture was spotted onto. Whatman p81 phosphocellulose filter paper (Whatman) and washed for 30 min with 0.2M sodium carbonate buffer (pH 9.2) at room temperature with 2-3 changes of the buffer, then washed briefly with acetone. The dried filters were counted in a liquid scintillation counter.

Metabolic Labelling

For ³²p labelling, cells were cultured in FCS free media for 2 days before incubation in a phosphate-free medium for 2 hr. Cells were then incubated in a phosphate-free medium containing 3 mCi of [³²P] orthophosphate (40 μCi/ml)(Amersham) for 30 min, and then, stimulated with IL-1β (1 ng/ml). The cultures were incubated for 6 hr at 37° C. in an atmosphere of 5% CO₂. Cells were collected and lysed with mild IP buffer. Immunoprecipitates of anti-CBP antibody were separated by electrophoresis on SDS-7% polyacrylamide gel and visualised by autoradiograph, or quantified by counting of excised radioactive bands.

Chromatin Immunoprecipitation (ChIP) Assay

A-549 cells were treated with IL-1β (1 ng/ml) in the presence or absence of various doses of dexamethasone as described above. After a 4-hr incubation, protein-DNA complexes were fixed by formaldehyde (1% final concentration) and treated as previously described (13). Cells were resuspended in 200 μl of SDS lysis buffer (50 mM Tris; pH 8.1, 1% SDS, 5 mM EDTA, complete proteinase inhibitor cocktail) and subjected to 3 steps with 10-sec pulses sonication on ice. Sonicated samples were centrifuged to spin down cell debris and the soluble chromatin solution were immunoprecipitated using sonicated salmon sperm DNA agarose A slurry (Upstate Biotechnology, Buckingham, UK) as described by Chen et al. (Chen, H et al (1999) Cell 98:675-686). Protein-bound immunoprecipitated DNA was washed with LiCl wash buffer and TE, and immune-complexes were eluted by adding elution buffer (1% SDS, 0.1M NaHCO₃). The elution was treated successively for 4 hr at 65° C. in 200 mM NaCl/1% SDS to reverse crosslinks and incubated for 1 hr at 45° C. with 70 μg/ml Proteinase K (Sigma). DNA extracted with phenol/chloroform, precipitated with ethanol/0.3M NaHCOOH/20 μg glycogen, and resuspended in 50 μl of TE. Quantitative PCR was performed with 10 μl of DNA sample and 30 cycles. Primer pairs of GM-CSF and SLPI were; GM-CSF forward 5-CTGACCACCTAGG GAAAAGGC-3, GM-CSF reverse 5-CAGCCACATCCTCCTCCAGAGAAC-3, SLPI forward 5-TCATAGCCTTACCTGGCATAG-3, SLPI reverse 5-TGGACTTCATGGTGAAGGCAG-3. PCR products were resolved by 3% agarose-gel and visualized with ethidium bromide.

Statistics

Results are expressed as means±standard error of the mean (SEM). A multiple comparison was made between the mean of the control and the means from each individual treatment group by Dunnett's test using SAS/STAT software (SAS Institute Inc., Cary, N.C., USA). All statistical testing was performed using a two-sided 5% level of significance. The concentrations of dexamethasone or trichostatin A producing 50% inhibition (IC₅₀) were calculated from concentration-response curves by linear regression.

Results

Evidence for a Role of Histone Acetylation in IL-1β and Dexamethasone-Induced Gene Expression

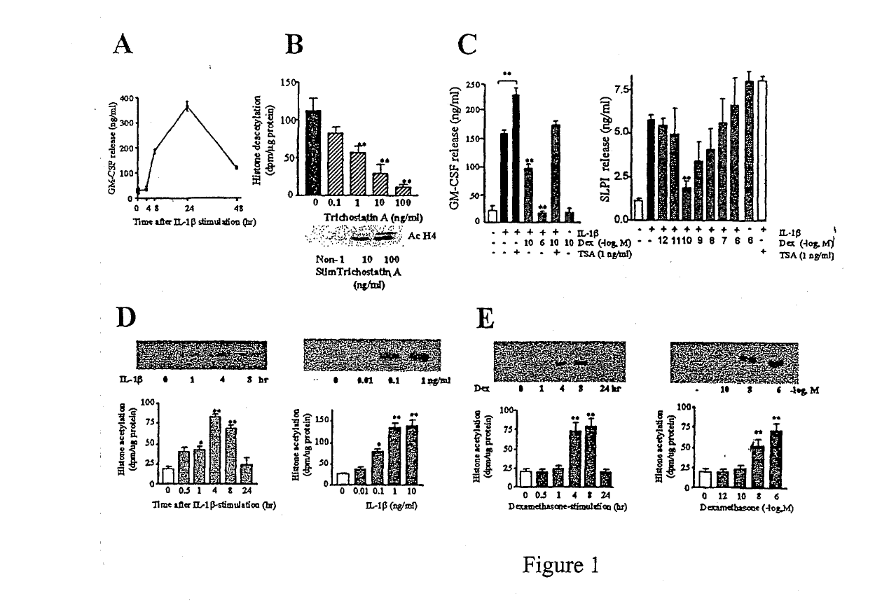

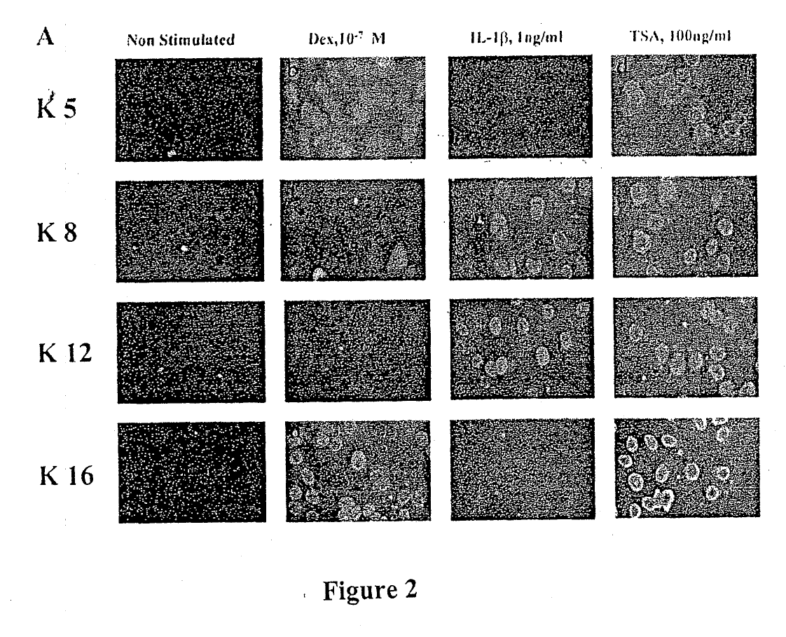

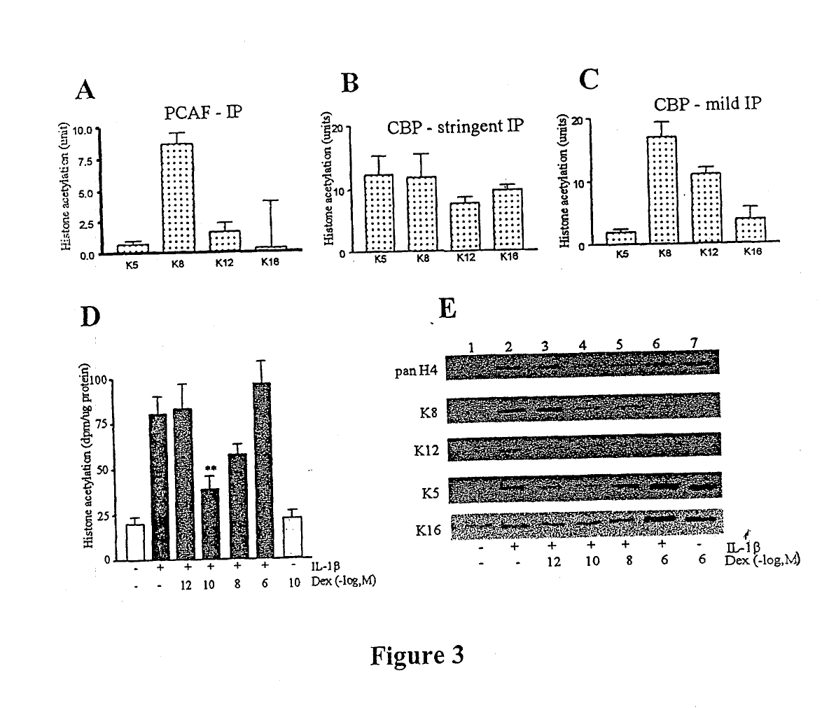

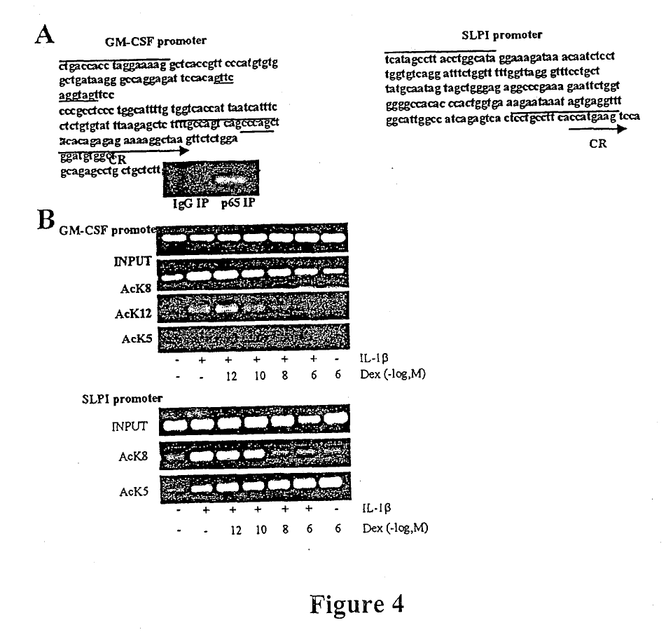

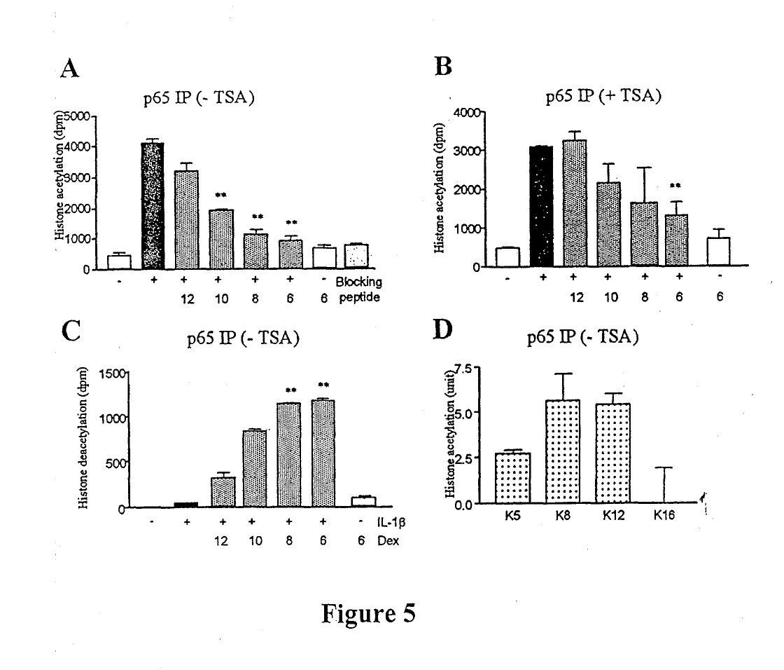

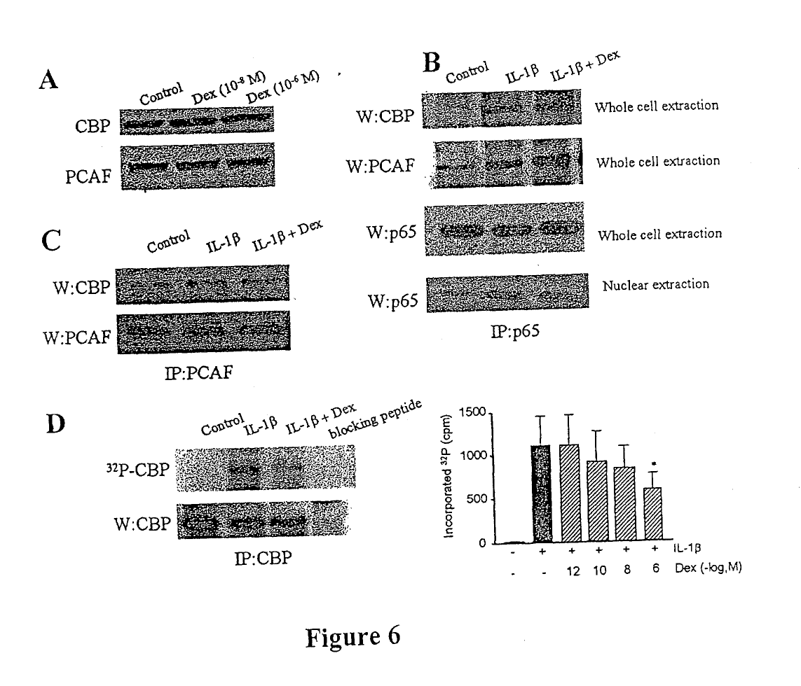

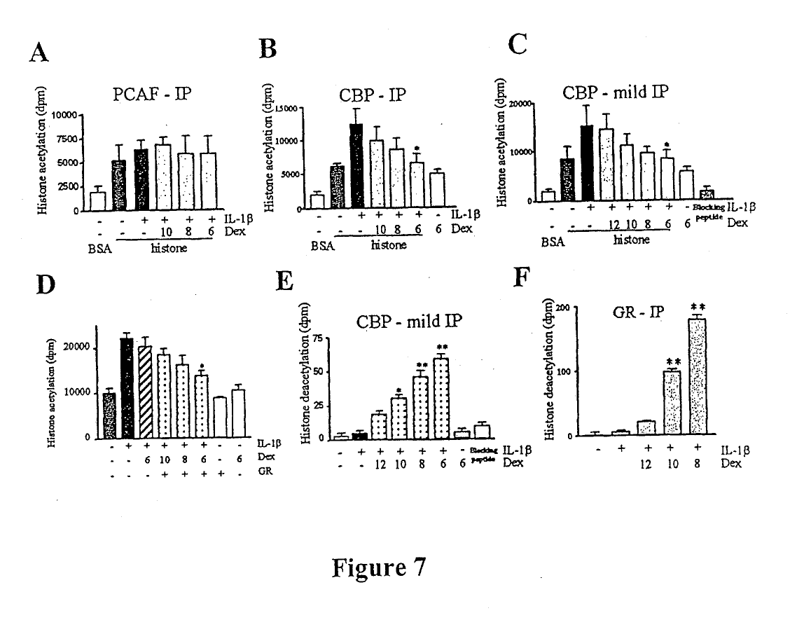

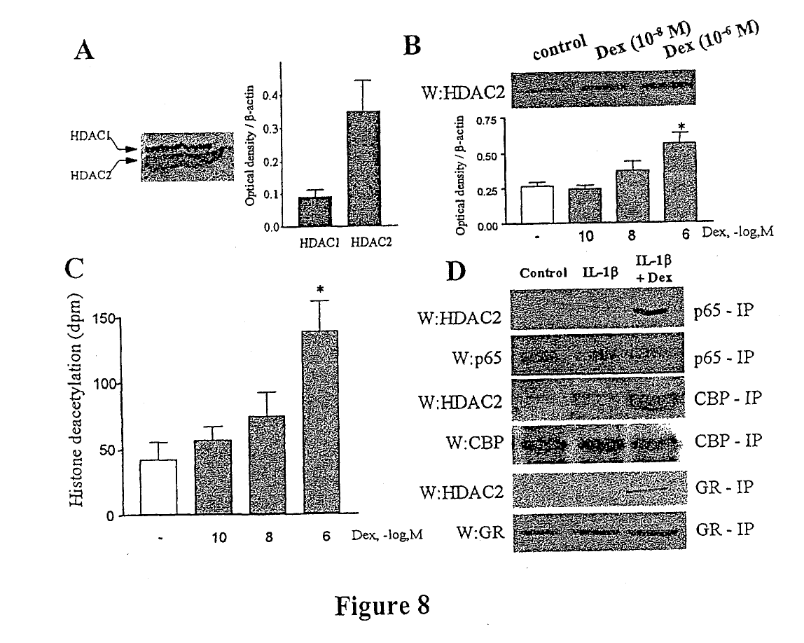

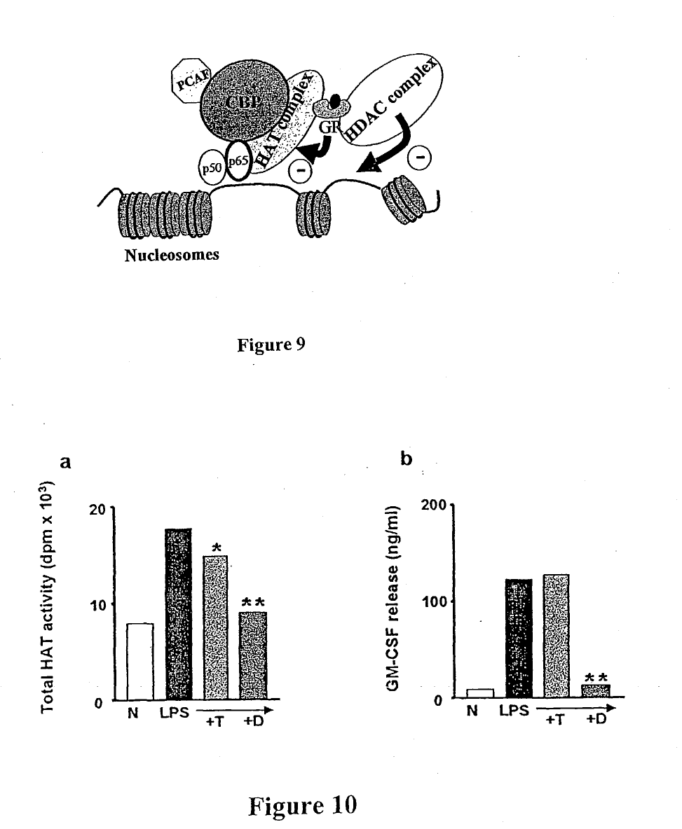

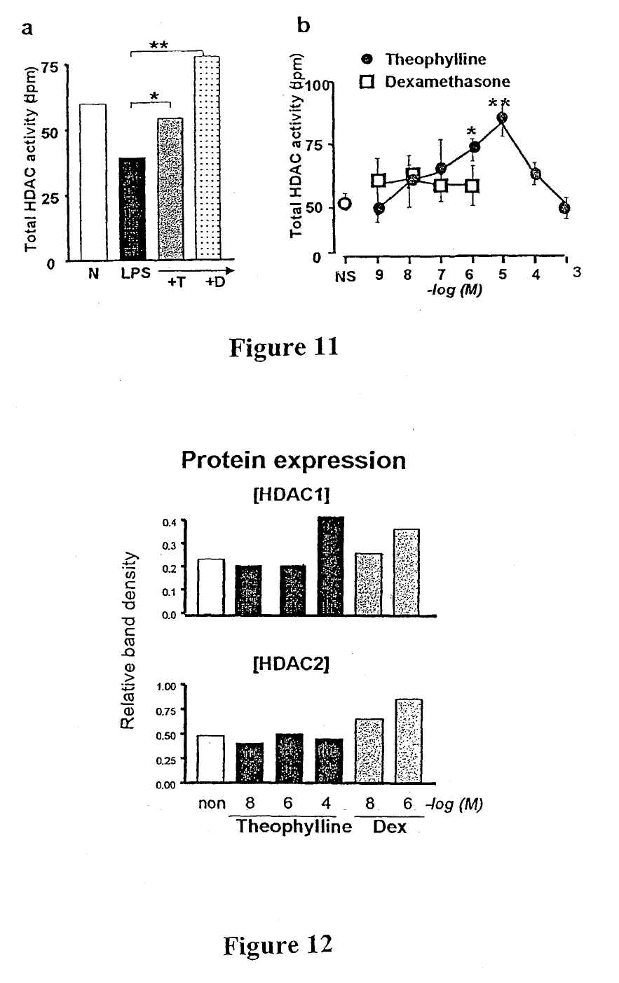

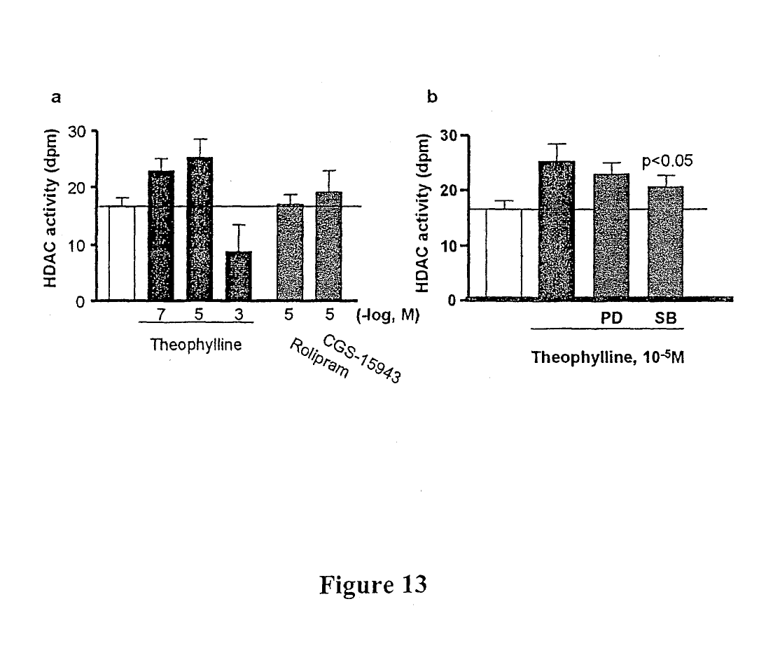

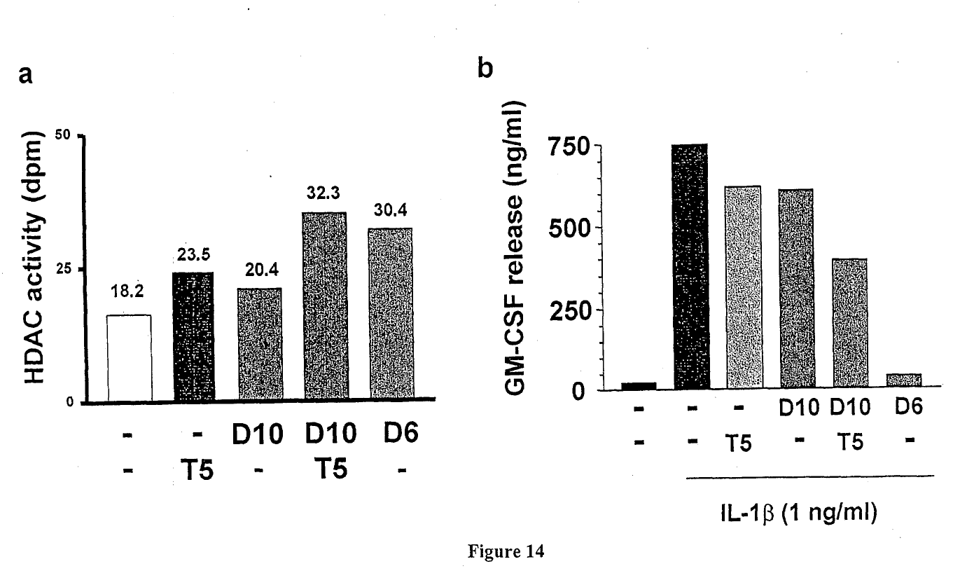

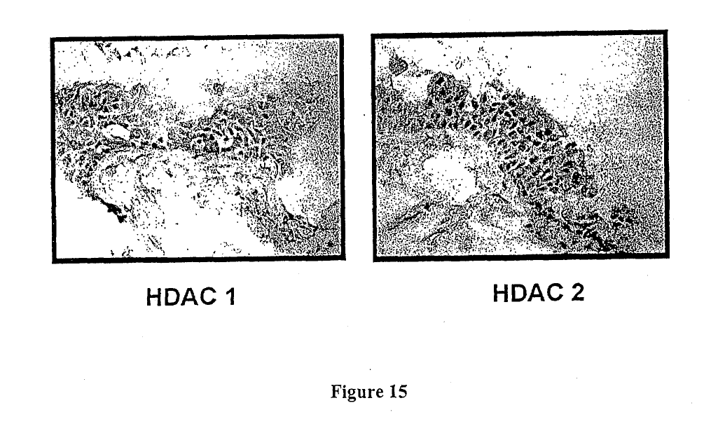

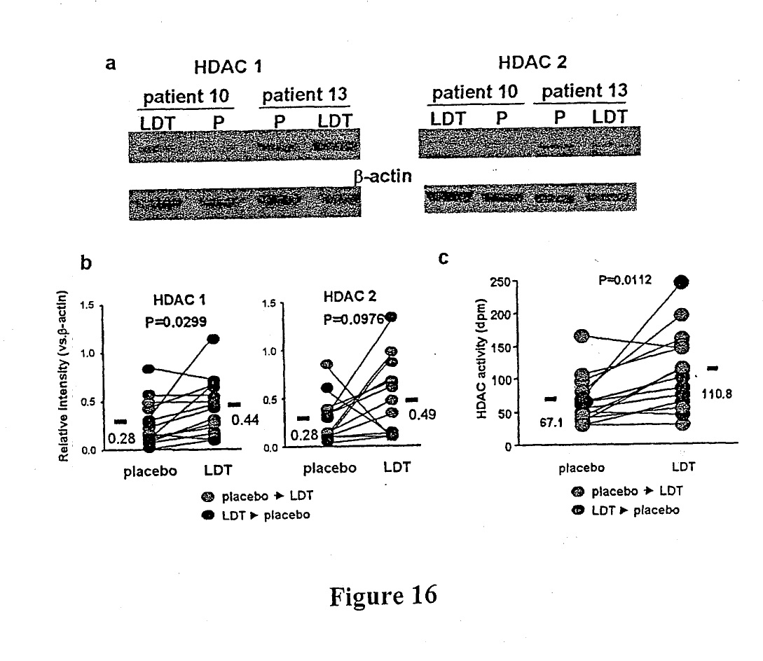

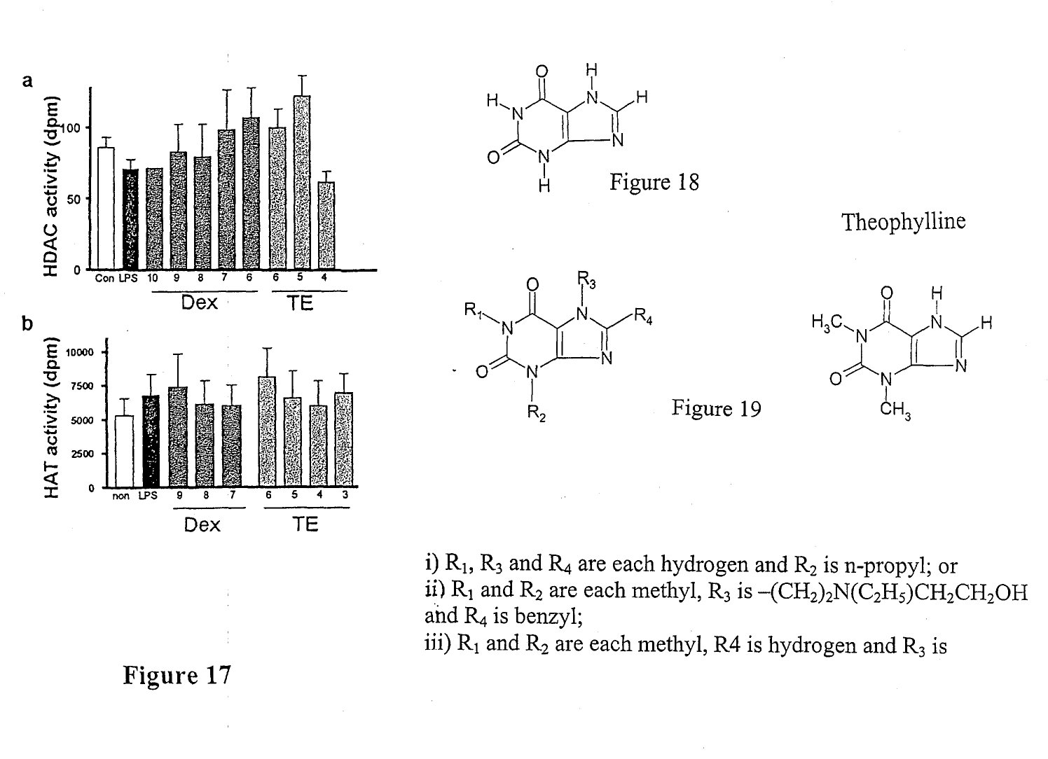

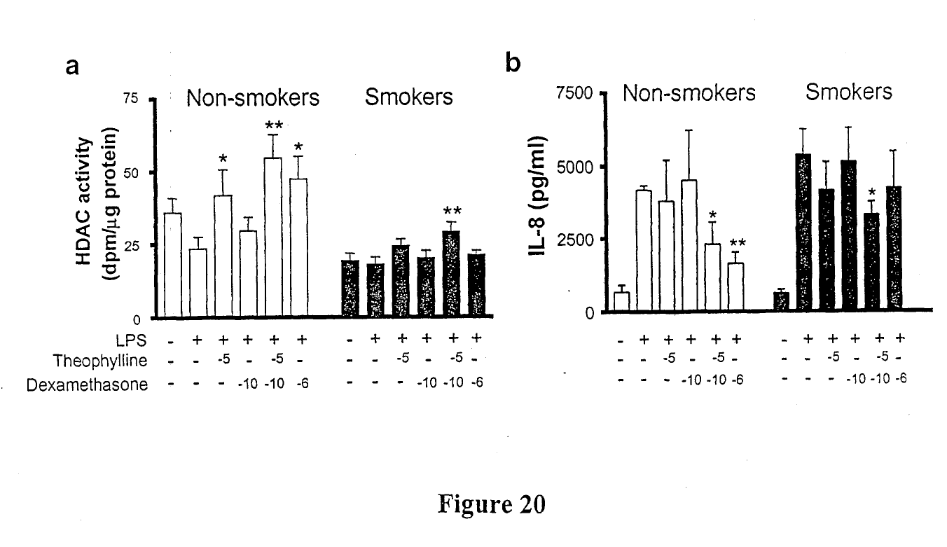

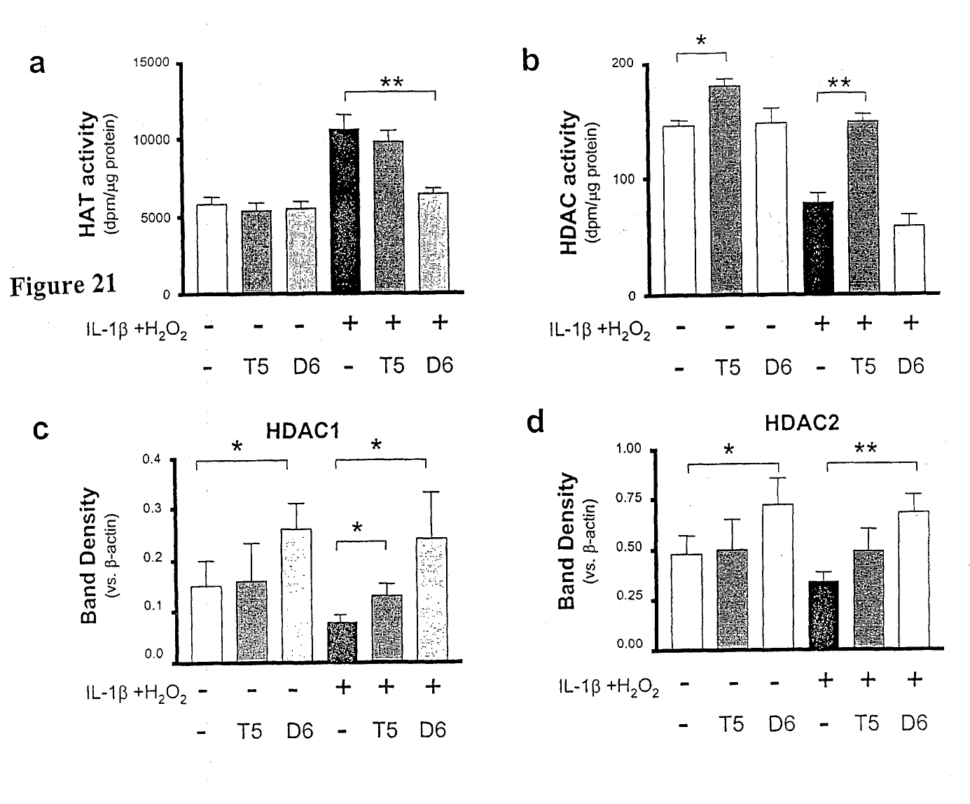

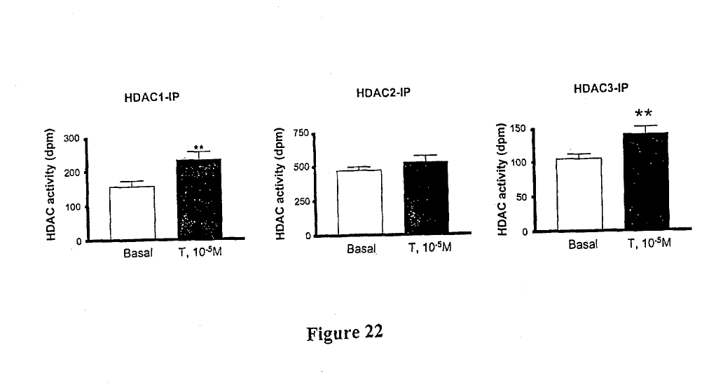

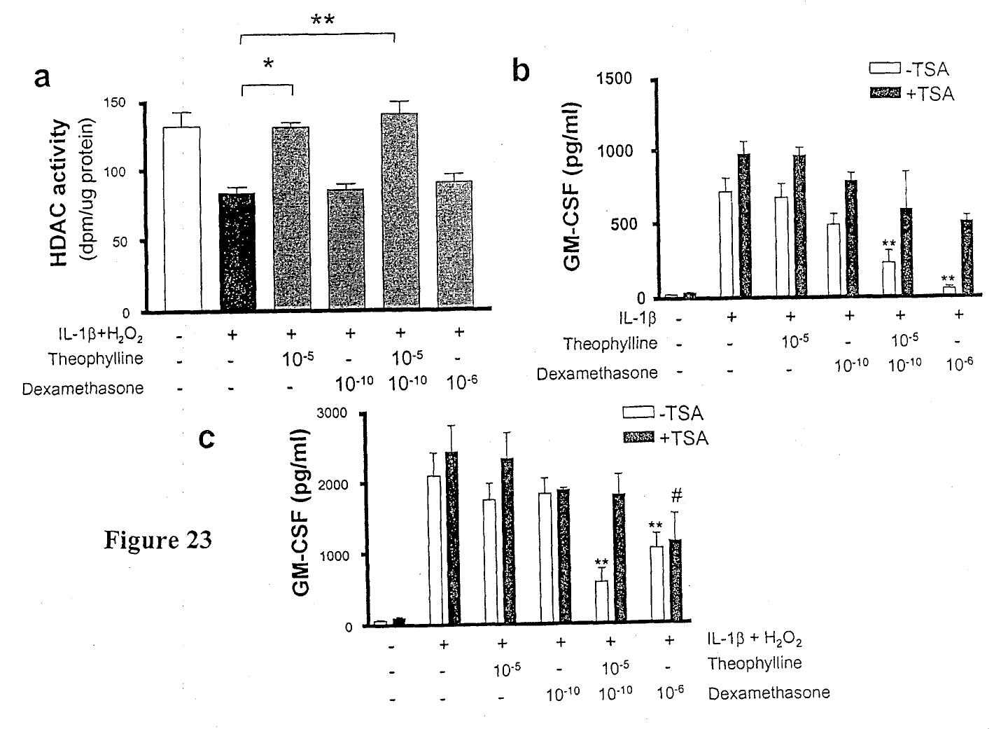

IL-1β (1 ng/ml) stimulated the production of GM-CSF (157±6 ng/ml) within the culture supernatant after 6 hr, whereas low levels of GM-CSF were found in the supernatant of control untreated cells (30±10 ng/ml). No induction of GM-CSF release was seen before 4 hours and a maximum was reached at 24 hours (FIG. 1A). The HDAC inhibitor, trichostatin A (TSA) gave a concentration-dependent decrease in HDAC activity in A549 cells (112±21 to 11±3 dpm/ng protein), with an IC₅₀ (1.1 ng/ml) similar to that previously reported (23). This was associated with a marked increase in histone acetylation as measured by [³H]-acetate incorporation and by Western blotting analysis (FIG. 1B). In addition, TSA (1 ng/ml) enhanced IL-1β-induced GM-CSF release (233±12 versus 157±6 ng/ml)(FIG. 1C).

IL-1β (1 ng/ml) increased SLPI production (6.0±0.5 versus 1.2±0.3 ng/ml) an effect which was further enhanced by pretreatment with TSA (1 ng/ml)(8.2±0.3 versus 6.0±0.5 ng/ml)(FIG. 1C).

Role of Histone Acetylation in Dexamethasone-Mediated Actions

We next investigated the effect of dexamethasone on IL-1β stimulated mediator release. Dexamethasone produced a concentration-dependent inhibition of IL-1β-stimulated GM-CSF release (IC₅₀: 2.3×10⁻⁹M) which was maximal at 10⁻⁶M (FIG. 1C). The inhibitory effect of dexamethasone on IL-1β-induced GM-CSF production was shifted 5-fold to the left in the presence of TSA (1 ng/ml)(IC₅₀=1.2×10⁻⁸M versus 2.3×10⁻⁹M), suggesting an involvement of HDACs in the inhibitory actions of dexamethasone (FIG. 1B). These results suggest a possible role for histone acetylation/deacetylation in the regulation of GM-CSF expression by dexamethasone.

Dexamethasone alone caused a concentration-dependent induction of SLPI (EC₅₀=0.9×10⁻⁸M) which reached a maximum at 1 μM (FIG. 1C). In contrast, dexamethasone had a biphasic effect on IL-1β-stimulated SLPI production with an initial decrease at 10⁻¹⁰M with a subsequent increase at higher concentrations (10⁻⁹ to 10⁻⁶M)(FIG. 1C). This data confirms that the ability of dexamethasone to inhibit IL-1β-stimulated gene transcription occurs at lower concentrations than those required to stimulate gene transcription.

Chromatin Acetylation is Associated with Transcriptional Activation by IL-1β and Dexamethasone

IL-1β caused both a time- and concentration-dependent 4-5-fold increase in histone acetylation in whole cell incorporation assays (FIG. 1D), which preceded GM-CSF production by IL-1β. This induction was maximal at 1 ng/ml (137±15 versus 25±3 dpm/μg protein) and was detectable 30 min after IL-1β-stimulation (41±6 versus 18±4 dpm/μg protein). The stimulation peaked between 4-8 hr and returned to control levels after 24 hr. TSA (1 ng/ml) enhanced both basal (162±21 versus 50±5 dpm/μg protein) and IL-1μ-stimulated (1543±143 versus 137±15 dpm/μg protein) histone acetylation. Dexamethasone also produced a time- and concentration-dependent increase in histone acetylation with a maximum induction between 4-8 hr at concentrations of 10⁻⁸ M or greater FIG. 1E). TSA (1 ng/ml) enhanced the basal (162±21 versus 20±5 dpm/μg protein) and dexamethasone-induced histone acetylation (984±50 versus 71±9 dpm/μg protein). In subsequent experiments, histone acetylation was measured at 6 hr following IL-1β (1 ng/ml) stimulation in the presence or absence of dexamethasone.

These results were confirmed by immunofluorescence and confocal microscopy (data not shown). This also showed that IL-1β but not dexamethasone or TSA, caused nuclear translocation of p65 whilst dexamethasone, but not IL-1β or TSA, enhanced GR nuclear translocation.

Specific Targeting of Histone H4 Lysine Residues by IL-1β and Dexamethasone

We determined the pattern of lysine acetylation following IL-1β and dexamethasone stimulation. Dexamethasone targeted acetylation on histone H4 lysines K5 (53±9% positive nuclei) and K16 (36±16% positive nuclei), whilst IL-1β acetylated K8 (42±15% positive nuclei) and K12 (37±4% positive nuclei). IL-1β (1 ng/ml) also produced a much weaker nuclear staining for acetylated K5 than that seen with dexamethasone (FIG. 2).

Acetylation of specific lysine residues is mediated through the HAT activities of co-activator molecules including CBP and PCAF. We therefore examined the possible role of CBP and PCAF in mediating IL-1β-stimulated acetylation of specific histone H4 lysine residues. Cells were stimulated with IL-1β for 6 hours before total cellular proteins were isolated. CBP and PCAF were immunoprecipitated under mild- or stringent-IP conditions to indicate whether the co-activators alone or their associated factors were involved in the acetylation of specific lysine residues. PCAF was able to stimulate predominantly K8 acetylation (FIG. 3A) confirming data from Schiltz and colleagues (Schiltz, R. L et al (1999) J. Biol. Chem. 274:1189-1192). In comparison, CBP isolated under stringent IP conditions was able to acetylate all histone H4 lysines (FIG. 3B). In contrast, CBP-complexes isolated using mild IP conditions predominantly acetylated K8 and K12 (FIG. 3C) confirming the immunocytochemistry results. This suggests that IL-1β may stimulate K8 and K12 acetylation through a CBP-associated HAT rather than directly through CBP alone.

Dexamethasone Targets IL-1β-Stimulated Acetylation of Histone H4 K8 and K12

We next examined whether IL-1β-stimulated K8 and K12 acetylation was a target for dexamethasone actions. Initial experiments were performed in whole cell extracts from cells treated with IL-1β in the presence or absence of increasing concentrations of dexamethasone. IL-1β induced a 4-fold increase in histone acetylation (FIG. 3D). Dexamethasone (10⁻¹⁰ M) alone had no effect on basal histone acetylation (23.7±4.1 dpm/μg protein). Dexamethasone had a biphasic effect on IL-1β-stimulated histone acetylation (FIG. 3D). Low concentrations of dexamethasone (10⁻¹⁰M) inhibited IL-1β-stimulated histone acetylation whilst higher concentrations of dexamethasone (10⁻⁸ and 10⁻⁶M) returned [³H] acetate incorporation to levels seen with IL-1β alone (FIG. 3D). TSA (100 ng/ml) caused a marked elevation of IL-1β-(1543±143 versus 71±9 dpm/μg protein) and IL-1β plus dexamethasone (10⁻¹⁰ M)(435±28 versus 37±5 dpm/μg protein)-stimulated histone acetylation to levels much greater than that seen with IL-1β treatment alone (71±9 dpm/μg protein).

Western analysis of specific acetylated lysines showed that dexamethasone inhibited IL-1β-stimulated K8 and K12 acetylation with almost total suppression at 10⁻¹⁰M (FIG. 3E, lane 4). In addition, the small induction of K5 acetylation by IL-1β was also suppressed at low (10⁻¹² and 10⁻¹⁰M) concentrations of dexamethasone (FIG. 3E, lanes 1 to 4) whereas at higher concentrations (10⁻⁸ and 10⁻⁶M) marked acetylation of K5 occurred (FIG. 3E, lanes 5 and 6). Dexamethasone also enhanced K16 acetylation at higher concentrations (10⁻⁸ and 10⁻⁶M). This data suggests that dexamethasone at low concentrations can inhibit histone acetylation induced by IL-1β whereas at higher concentrations dexamethasone can itself induce histone acetylation at specific target lysine residues.

IL-1β Increases K8 and K12 Acetylation Associated with the GM-CSF Promoter

The previous data examined gross histone acetylation. It was essential therefore to determine whether the interaction of the p65 activated HAT complex with GR occurs specifically on the GM-CSF and SLPI promoters. We analysed the nucleosomal events involved in GM-CSF transactivation by semi-quantitative chromatin immunoprecipitation. This technique is based on the crosslinking of protein/DNA and protein/protein complexes within the cell by formaldehyde treatment, followed by chromatin sonication, immunoprecipitation with specific antibodies, and precise quantification of the immunoprecipitated DNA segments by PCR. This procedure quantitatively assesses the in vivo association of a given protein to a defined DNA region. Two different genomic sites were investigated: the GM-CSF (−191-+10) and the SLPI promoter (−170-+32)(FIG. 4A). PCR amplifications were carried out on a fixed amount of immunoprecipitated DNA, followed by 30 cycles of PCR with the appropriate primer pairs. Analysis of protein interactions at the selected regions was performed in A549 cells after treatment with IL-1β and/or dexamethasone. Initial studies indicated that following IL-1β treatment p65 immunoprecipitates showed a marked enrichment of GM-CSF promoter DNA (FIG. 4A). Immunoprecipitation with an antibody against acetylated K8 or K12 resulted in the enrichment for the DNA segments encompassing the GM-CSF promoter following IL-1β treatment (FIG. 4B). These data demonstrate that p65-mediated activation of the GM-CSF promoter in vitro is concomitant with the acetylation of histone H4 K8 and K12 residues. Increasing concentrations of dexamethasone caused a reduction in the enrichment of acetylated K8- and K12-associated GM-CSF promoter fragments (FIG. 4B). This effect correlated well with dexamethasone repression of GM-CSF release. In contrast, acetylated K5 residues were not associated with the GM-CSF promoter segment either at baseline or following IL-1β treatment (FIG. 4B). Immunoprecipitation with an antibody against acetylated K8 resulted in the enrichment for the DNA segments encompassing the SLPI promoter following IL-1β treatment. IL-1β stimulation of cells had no effect on K5-associated SLPI promoter DNA. In contrast, dexamethasone caused a concentration-dependent increase in K5-associated DNA enrichment in both basal and IL-1β-treated cells (FIG. 4B).

This data indicates that histone acetylation induced by IL-1β or dexamethasone occurs on specific lysine residues associated with distinct pro- and anti-inflammatory genes.

Effect of Dexamethasone on p65-Induced Histone Acetylation and Deacetylation

In order to clarify the inhibitory mechanism of dexamethasone on histone acetylation, we investigated p65-associated histone acetylation and deacetylation in IL-1β stimulated cells in the presence or absence of increasing concentrations of dexamethasone. In some experiments the role of histone deacetylases on dexamethasone action was examined by pre-treating the cells with TSA (100 ng/ml). Whole cell lysates were made and p65 immunoprecipitates isolated under mild IP conditions (see methods) examined for associated histone acetylation and deacetylation activity (FIG. 5). In these p65 immunoprecipitation experiments histone acetylation was increased 9-10 fold following IL-1β stimulation (FIG. 5A). Dexamethasone inhibited p65-associated IL-1β-induced histone acetylation in a concentration-dependent manner (IC₅₀=4×10⁻¹⁰M). Dexamethasone alone produced no change in p65-associated histone acetylation from that seen in control untreated samples (FIG. 5A). Control experiments with anti-p65 antibody blocking peptide showed no histone acetylation (221±122). TSA (100 ng/ml) caused a 50-fold shift in the dexamethasone concentration-response curve (IC₅₀ value; 9×10⁻⁸M versus 4×10⁻¹⁰M)(FIG. 5B) suggesting that the inhibitory effects of dexamethasone require some HDAC involvement. In the same immunoprecipitates, dexamethasone enhanced histone deacetylation in a concentration-dependent manner (FIG. 5C). To confirm that the p65-IPs were acetylating the same lysine residues as IL-1β, p65-IPs were examined for specific forms of acetylated histone H4 lysines by ELISA. The p65-IPs targeted mainly K8 and K12 acetylation, with a smaller effect on K5 acetylation (FIG. 5D). This data confirmed the results seen by immunocytochemistry and CBP immunoprecipitates isolated under mild IP conditions following IL-1β stimulation (see FIGS. 2 and 3C).

Effect of Dexamethasone on Co-Activator Expression, Association with p65 and Phosphorylation

A number of co-activators may be involved in IL-1β-stimulated induction of histone acetylation and its subsequent amelioration by dexamethasone (Fontes, J. D et al (1999) Mol. Cell Biol. 19:941-947; Kamei, Y et al (1996) Cell 85:403-414; Perkins, N. D et al (1997) Science 275:523-527; Sheppard, K. A et al (1998) J. Biol. Chem. 273:29291-29294). Initially we examined the effect of dexamethasone on CBP and PCAF expression. Dexamethasone (10⁻⁸ and 10⁻⁶M, 6 hrs) had no effect on CBP or PCAF expression ruling out a reduction in CBP or PCAF expression as a mechanism for inhibiting IL-1β-stimulated histone acetylation (FIG. 6A). An alternative mechanism of dexamethasone action could be to reduce the interaction between the IL-1β-stimulated NF-κB p65 subunit and CBP or PCAF. Using p65-IPs followed by Western blotting there was no difference in the ability of IL-1β to enhance p65/CBP or p65/PCAF interactions within the nucleus following dexamethasone (10⁻⁶M) treatment (FIG. 6B). Furthermore, dexamethasone did not inhibit p65 translocation (FIG. 5B) or IL-1β-induced CBP/PCAF association (FIG. 6C).

Inhibition of phosphorylation by MAPK pathways by dexamethasone has been proposed to play an important role in glucocorticoid actions (Caelles, C et al (1997) Genes Dev. 11:3351-3364; Rider, L. G et al (1996) J. Immunol. 157:2374-2380; Swantek, J. L et al (1997) Mol. Cell Biol. 17:6274-6282). These pathways may also regulate CBP activation by transcription factor phosphorylation or a direct effect on CBP, potentially altering histone acetylation and transactivation capabilities (Espinos, E et al (1999) Mol. Cell Biol. 19:3474-3484). IL-1β significantly induced immunoprecipitated CBP phosphorylation which was inhibited by dexamethasone (10⁻⁶M)(FIG. 6D). However, concentrations of dexamethasone which repressed IL-1β-stimulated gene expression and histone acetylation had no effect on CBP phosphorylation suggesting that although higher concentrations of dexamethasone can indeed inhibit CBP phosphorylation this effect does not account for the repression of histone acetylation by dexamethasone. Direct acetylation has been shown to be important in the activity of some transcription factors and co-activators (Boyes, J et al (1998) Nature 396:594-598; Gu, W. & R. G. Roeder (1997) Cell 90:595-606; Imhof, A. & A. P. Wolffe (1998) 8:R422-R424. However, there was no acetylation of CBP or PCAF in these cells following either IL-1β or dexamethasone treatment (data not shown).

Effect of Dexamethasone on Co-Activator-Associated Histone Acetylation

It has previously been shown that PCAF acetylates H4 K8 only (see FIG. 3A and Schiltz, R. L et al (1999) J. Biol. Chem. 274:1189-1192 and our data showing IL-1β-induced acetylation of K8 and K12 suggests that PCAF alone is unlikely to mediate IL-1β-induced histone acetylation. Further evidence for a lack of a role for PCAF was suggested by a failure of cells treated with IL-1β to show enhanced immunoprecipitated PCAF histone acetylase activity or for cells co-incubated with increasing concentrations of dexamethasone to modify immunoprecipitated PCAF activity (FIG. 7A).

We have earlier shown that IL-1β stimulated a CBP-associated HAT activity. We wished to investigate whether this CBP-associated activity was a target for dexamethasone activity. Cells were stimulated with IL-1β (1 ng/ml) for 6 hours in the presence or absence of increasing concentrations of dexamethasone. CBP was immunoprecipitated from the cells under mild or stringent conditions (see methods) and histone acetylation assays performed after the addition of exogenous histones. IL-1β caused an elevation in CBP-dependent histone acetylation under both stringent and mild IP conditions (FIGS. 7B & C). This activity peaked at 4 hr and returned to baseline by 24 hr (data not shown). Dexamethasone caused a concentration-dependent reduction in IL-1β-stimulated CBP-dependent histone acetylation (IC₅₀=8×10⁻⁹M) (FIG. 7B). Dexarmethasone alone did not inhibit basal immunoprecipitated CBP-associated histone acetylation (FIG. 7B).

Under stringent IP conditions IL-1β causes acetylation of all histone H4 lysine residues in contrast to the K8 and K12 pattern seen with CBP immunoprecipitated under mild IP conditions. Using CBP-extracted under mild IP conditions, IL-1β-induced elevation in CBP-associated histone acetylation was also inhibited by dexamethasone (FIG. 7C). CBP isolated under these conditions was more sensitive to the inhibitory effects of dexamethasone than those seen with CBP isolated using more stringent IP conditions (IC₅₀; 4×10⁻¹¹ versus 8×10⁻⁹M). Again dexamethasone alone did not inhibit basal CBP-associated histone acetylation. These results suggest that although repression of CBP may account for some of the repressive effect of dexamethasone on IL-1β-stimulated histone acetylation, it is not responsible alone for the repression of histone acetylation by dexamethasone and that CBP-associated co-factors are more sensitive to dexamethasone repression. Additionally, failure of CBP to induce histone acetylation at the higher concentrations of dexamethasone suggests that CBP in isolation does not mediate dexamethasone-induced histone acetylation.

In order to confirm that this inhibitory action of dexamethasone was mediated via GR, we performed HAT assays using immunoprecipitated CBP from IL-1β-treated cells and highly purified exogenous GR (1 ng/ml). These experiments were conducted in the presence of TSA (100 ng/ml) in order to inhibit endogenous HDAC activity that may otherwise interfere with the interpretation of the data. IL-1β (1 ng/ml) caused a marked increase in histone acetylation (FIG. 7D). Dexamethasone alone, in the absence of exogenous GR, had no effect on IL-1β-stimulated histone acetylation. In addition, the isolated GR complex showed no histone acetylation activity in the presence or absence of CBP-immunoprecipitate (FIG. 7D). The dexamethasone-GR complex inhibited IL-1β-stimulated CBP-mediated histone acetylation in a concentration dependent manner (FIG. 7D). This data suggests that in the absence of HDAC activity dexamethasone, acting through GR, is able to suppress CBP-associated histone acetylation.

The CBP-associated complex immunoprecipitated under mild IP conditions showed no increase in histone deacetylase activity after IL-1β treatment alone (FIG. 7E). However, with increasing concentrations of dexamethasone the levels of HDAC activity were markedly enhanced reflecting either induction of HDAC or recruitment of HDAC to the CBP immunoprecipitated complex (FIG. 7E). GR immunoprecipitates from both non-stimulated and IL-1β-stimulated cells did not show any histone deacetylation activity (FIG. 7F). In contrast, treatment with dexamethasone induced a concentration-dependent increase in histone deacetylation (FIG. 7F). These experiments showed that GR was associated with a histone deacetylase activity which was induced in a concentration dependent manner by dexamethasone. This induction reached significant levels at the concentrations which inhibited GM-CSF release and histone acetylation (FIG. 7F and FIGS. 1C & 3D).

Effect of Dexamethasone on HDAC Expression, Activity and Recruitment

We have shown that dexamethasone induced histone deacetylation in GR-, p65- and CBP-immunoprecipitates. Furthermore, TSA decreased the inhibitory effect of dexamethasone on IL-1β-induced GM-CSF production, histone acetylation and immunoprecipitated p65-associated histone acetylation. These results suggest that HDACs are involved in the inhibitory effects of dexamethasone. We, therefore, determined the effect of dexamethasone on HDAC expression, histone deacetylase activity and p65/HDAC association. A549 cells expressed mainly HDAC2 and very little HDAC1 (FIG. 8A). Dexamethasone induced both HDAC2 expression and histone deacetylation (FIGS. 8B & C) but the concentration at which dexamethasone induced these effects (10⁻⁶ M) was greater than that which repressed IL-1β-stimulated histone acetylation (10⁻¹⁰ M) (see FIG. 3D). This suggests that dexamethasone repression of IL-1β-stimulated histone acetylation was not due to induction of newly synthesised HDAC protein or activity. We, therefore, examined HDAC2 association with the activated HAT complexes following incubation of cells with IL-1β and low doses of dexamethasone. Western blot analysis of p65-immunoprecipitates showed a recruitment of HDAC2 to the p65 immunoprecipitated complex following treatment of cells with IL-1β and low concentration (10⁻¹⁰M) of dexamethasone (FIG. 8D), suggesting a role for HDAC2 in the suppressive actions of dexamethasone. Similarly, Western blot analysis of CBP- and GR-IPs also showed a recruitment of HDAC2 to the GR IP complexes (FIG. 8D).

Discussion

IL-1β caused a concentration-dependent increase in GM-CSF expression which was inhibited by dexamethasone at concentrations 5-10-fold lower than those which caused transactivation of SLPI. The effect of the HDAC inhibitor TSA suggested that histone acetylation status may play a role in the regulation of GM-CSF and SLPI release. Increased gene expression by both IL-Iβ and dexamethasone were associated with increases in histone H4 acetylation status. IL-1β specifically caused acetylation of histone H4 K8 and K12 and weakly acetylated K5 whilst dexamethasone markedly acetylated K5 and K16, with no effect on K8 and K12. Dexamethasone repressed IL-1β-induced GM-CSF expression and K8 and K12 acetylation at 5-10-fold lower concentrations than that which induced histone acetylation/deacetylation or SLPI induction. Using chromatin immunoprecipitation assays we confirmed that the differential acetylation of lysine residues by IL-1β and dexamethasone did not occur purely at the gross histone level but also occurred at both the GM-CSF and SLPI promoters. TSA attenuated the inhibitory effect of dexamethasone on GM-CSF production and histone acetylation suggesting a role for HDACs in dexamethasone actions.

Previous studies have shown a role for CBP in mediating NF-κB-driven gene transcription (Gerritsen, M. E et al (1997) Proc. Natl. Acad. Sci. U.S.A. 94:2927-2932) and more recent studies have shown that overexpression of CBP can modulate GR cross-talk with NF-κB (Perkins, N. D et al (1997) Science 275:523-527; Sheppard, K. A et al (1998) J. Biol. Chem. 273:29291-29294). The pattern of histone acetylation induced by CBP/p300 and PCAF are distinct, both from each other, and from those found in the present study following stimulation by IL-1β or dexamethasone (Schiltz, R. L et al (1999) J. Biol. Chem. 274:1189-1192). CBP is able to acetylate all the relevant lysine residues of histone H4 (Kimura, A. & M. Horikoshi (1998) FEBS Lett 431:131-133) suggesting that CBP is the most likely target for competition between GR and p65, or indeed other transactivating proteins in these cells. CBP has several transactivating domains and the specific domain used varies from one promoter to another and may direct acetylation of specific histone residues (Martinez-Balbas, M. A et al (1998) EMBO J. 17:2886-2893). CBP regulates the lysine residues acetylated by both IL-1β and dexamethasone, however, the targeting of specific lysine residues requires the association of additional co-activators, but not p300 or PCAF, which modulate CBP-mediated histone acetylation.

Our results suggest that the site of cross-talk between p65 and GR occurs at the level of regulation of histone H4 acetylation by CBP and HDAC2. Previous data has suggested a role for CBP and SRC-1 in the nuclear integration of NF-κB and GR actions (Sheppard, K. A et al (1998) J. Biol. Chem. 273:29291-29294). In this model it was proposed that competition for limiting amounts of CBP, or other co-activators, resulted in an inhibition of NF-κB driven gene transcription by GR. These studies used overexpression of CBP in order to overcome the actions of GR on NF-κB-mediated gene transcription. In contrast, our data shows no evidence for squelching as a mechanism for GR inhibition of IL-1β actions at least during the short (6 hr) time course of these experiments (Fontes, J. D et al (1999) Mol. Cell Biol. 19:941-947; Kamei, Y et al (1996) Cell 85:403-414; Perkins, N. D et al (1997) Science 275:523-527; Sheppard, K. A et al (1998) J. Biol. Chem. 273:29291-29294). However, exposure of cells for longer periods of time (24-48 hrs) to budesonide, a glucocorticoid agonist, indicates a time- and concentration-dependent reduction in CBP and RNA polymerase II expression (I A and Y. Nasuhara, unpublished observations).

Other studies have suggested that binding of GR to CBP disrupts the CBP/PCAF co-activation complexes (Korzus, E et al (1998) Science 279:703-707). We found no evidence that dexamethasone blocked IL-1β-stimulated p65/CBP and p65/PCAF association or the association between CBP and PCAF. Furthermore, our results fail to indicate a major role for PCAF in mediating IL-1β-dependent acetylation of lysines. In contrast, we have shown a direct effect of GR on inhibiting IL-1β-induced CBP complex-mediated histone acetylation. The histone acetylation of CBP immunoprecipitates extracted under mild immunoprecipitation conditions, in which a large number of other proteins were co-immunoprecipitated, was repressed by low concentrations of dexamethasone, and was specific to K8 and K12. Our results in which the CBP-associated complex, but not CBP alone, showed specificity for K8 and K12 indicates that other HATs as well as CBP are likely to be involved. Alternatively, HATs may interact with one another within a complex to modify the histone target lysines of each specific HAT. As these inhibitory effects of dexamethasone were decreased in the presence of TSA, HDACs were also indicated as playing a role in dexamethasone repression. However, this was not related to the induction of newly synthesised HDAC protein and activity but reflected recruitment of HDAC2 to a p65/CBP complex by GR.

Inhibition of MAPK phosphorylation by dexamethasone has been suggested to play an important role in glucocorticoid actions (Caelles, C et al (1997) Genes Dev. 11:3351-3364; Rider, L. G et al (1996) J. Immunol. 157:2374-2380; Swantek, J. L et al (1997) Mol. Cell Biol. 17:6274-6282). These pathways also play a role in CBP activation by phosphorylation of transcription factors, such as NF-κB and AP-1, and may also directly phosphorylate CBP, thereby altering transactivation. Although we demonstrated that IL-1β induced phosphorylation of CBP and that this could be inhibited by dexamethasone, the concentration of dexamethasone at which this reduction occurrs (10⁻⁶M) is greater than that which inhibited histone acetylation and inflammatory gene expression indicating that this mechanism of glucocorticoid action is less important for the anti-inflammatory actions of dexamethasone. In addition, we found no evidence for acetylation of CBP by either IL-1β or dexamethasone in these studies.

In summary, we have shown that both dexamethasone and IL-1β stimulated histone acetylation but each showed a different pattern of histone H4 acetylation. Low concentrations of dexamethasone (10⁻¹⁰M) which repress IL-1β-stimulated GM-CSF expression also repress IL-1β-stimulated CBP-associated histone acetylation at the GM-CSF promoter. Our data suggests that the activated GR complex inhibits acetylation of K8 and K12, by acting both as a direct inhibitor of CBP-associated histone acetylation and by recruiting HDAC2 to the p65/CBP HAT complex. This action does not involve de novo synthesis of HDAC protein or activity, or increased expression of CBP or PCAF. Thus, we found that both HAT and HDAC activities co-exist within same complex in the presence of p65 and GR and that they can each act independently without competing with each other (see FIG. 8). This mechanism for glucocorticoid repression is novel and establishes that inhibition of histone acetylation is an additional level of control of inflammatory gene expression. This further suggests that pharmacological manipulation of specific histone acetylation status is a potentially useful approach for the treatment of inflammatory diseases. Identification of the precise mechanism by which activated GR recruits HDAC2 may reveal new targets for the development of drugs that may dissociate the anti-inflammatory actions of glucocorticoids from their side effects which are largely due to gene induction.

### EXAMPLE 2

A Novel Molecular Mechanism of Action for Theophylline: Induction of Histone Deacetylase Activity

Clinically theophylline alone has limited anti-inflammatory actions but is an effective add-on therapy to corticosteroids in the treatment of asthma. Corticosteroids act, at least in part, by recruitment of histone deacetylases (HDACs) to the site of active gene transcription and thereby inhibiting the acetylation of core histones that is necessary for inflammatory gene transcription, as discussed in Example 1. We show both in vitro and in vivo that theophylline enhances HDAC activity in epithelial cells. This increased HDAC activity is then available for corticosteroid recruitment and predicts a co-operative interaction between corticosteroids and theophylline. This mechanism occurs at therapeutic concentrations of theophylline and is dissociated from phosphodiesterase (PDE) inhibition (the mechanism of bronchodilatation) or blockade of adenosine receptors, which are responsible for its side effects. Thus we have shown that theophylline exerts a novel anti-asthma effect through increasing HDAC activation which is subsequently recruited by corticosteroids to suppress inflammatory genes.

Materials and Methods

Patients: Fifteen mild stable asthmatic subjects (Table 1) receiving treatment with only the inhaled β₂-adrenergic agonist aerosol, albuterol, for intermittent relief of wheeze were recruited. All patients demonstrated a >15% improvement in FEV₁ following 200 μg of albuterol and airway hyperresponsiveness to methacholine with a provocative concentration of methacholine producing a 20% fall in FEV₁ (PC₂₀) of <4 mg/ml. All patients were atopic as defined by two or more positive skin prick tests to common allergens. None of the subjects studied had received oral or inhaled corticosteroids for the preceding twelve months or any other treatment apart from inhaled β₂ agonists. Current smokers or ex-smokers of more than five pack years and patients with FEV₁ less than 80% predicted were excluded.

TABLE 1
Clinical Features of subjects

| | Baseline | Placebo | Theophylline |
|---|---|---|---|
| Age (yrs) | 30.5 ± 2.1 | | |
| M/F | 8/7 | | |
| FEV₁ | 3.32 ± 0.14 | 3.27 ± 0.16 | 3.42 ± 0.09 |
| FEV₁ (% predicted) | 87.9 ± 8.1% | 86.3 ± 3.1 | 92.8 ± 2.6 |
| PC₂₀ methacholine | 0.85 ± 0.34 | 0.98 ± 0.47 | 1.2 ± 0.35 |
| PEF am | 471 ± 18 | 456 ± 14 | 469 ± 19 |
| NO | 20.6 ± 2.7 | 24.7 ± 2.6 | 23.2 ± 3.8 |
| Blood Theophylline (mg/ml) | | <1 | 4.3 ± 0.85 |
| BAL eosinophils (%) | | 3.4 ± 0.47 | 1.7 ± 0.28 |
| Muscosal eosinophils | | 1.83 ± 0.48 | 1.19 ± 0.43 |

FEV₁: Forced expiratory volume in 1 second

PC₂₀ methacholine: Concentration of methacholine that causes a 20% fall in FEV₁

Study design: The study was a 14-week double-blind randomised cross-over study comparing the effects of low dose theophylline (Euphylong: 800 μg twice daily), to that of placebo. Each treatment was administered for 5 weeks, separated by a four-week wash-out phase. All patients were reviewed at day 28, spirometry and airway responsiveness to methacholine were measured. At day 35, of each treatment period, venous blood was drawn for the measurement of serum theophylline and fiberoptic bronchoscopy and bronchoalveolar lavage were performed (John, M et al (1998) Am. J. Respir. Crit. Care Med. 157:256-262). The Royal Brompton Hospital Ethics Committee approved the study and all patients gave their informed consent.

Fibreoptic bronchoscopy and isolation of BAL macrophages: Subjects attended our bronchoscopy suite at 8.30 am after having fasted from midnight and were pretreated with atropine (0.6 mg iv) and midazolam (5-10 mg iv). Oxygen (31/min) was administered via nasal prongs throughout the procedure and oxygen saturation was monitored with a digital oximeter. Using local anaesthesia with lidocaine (4%) to the upper airways and larynx, a fibreoptic bronchoscope (Olympus BF10) was passed through the nasal passages into the trachea. Bronchoalveolar lavage (BAL) was performed from the right middle lobe using warmed 0.9% NaCl with 4 successive aliquots of 60 mls of 0.9% NaCl. BAL cells were spun (500 g; 10 min) and washed twice with Hanks buffered salt solution (HBSS) (John et al (1998)). Cytospins were prepared and stained with May-Grunwald stain for differential cell counts. Cell viability was assessed using trypan blue exclusion. In some experiments macrophages were isolated by plastic adhesion and cells (1×10⁶) incubated in 24 well plates in the presence of theophylline, dexamethasone or LPS (3 ng/ml).

Cell Culture: A549 cells were grown to 50% confluence in Dulbecco's modified medium (DMEM) containing 10% foetal calf serum (FCS) before incubation for 48-72 hr in serum-free media. Cells were stimulated by lipopolysaccharide (LPS, 3 ng/ml) in the presence of theophylline or dexamethasone.

GM-CSF ELISA: Determination of GM-CSF expression was measured by sandwich ELISA (Pharmingen, Lugano, Switzerland) according to the manufacturer's instructions.

Direct histone extraction: Histones were extracted from nuclei overnight using HCl and H2SO4 at 4° C. using a modified method from that as described by Turner and by Yoshida (Turner, B. M. & G. Fellows (1989) Eur. J. Biochem. 179:131-139; Yoshida, M et al (1990) J. Biol. Chem. 265:17174-17179). Cells were microfaged for 5 min and the cell pellets extracted with ice-cold lysis buffer (10 mM Tris-HCl, 50 mM sodium bisulphite, 1% Triton X-100, 10 mM MgCl2, 8.6% sucrose, complete protease inhibitor cocktail (Boehringer-Mannheim, Lewes, UK) for 20 min at 4° C. The pellet was repeatedly washed in buffer until the supernatant was clear (centrifuge at 8000 rpm, 5 min after each wash) and the nuclear pellet washed in nuclear wash buffer (10 mM Tris-HCl, 13 mM EDTA) and resuspended in 50 μl of 0.2N HCl and 0.4N H2SO4 in distilled water. The nuclei were extracted overnight at 4° C. and the residue microfuged for 10 min. The supernatant was mixed with 1 ml ice-cold acetone and left overnight at −20° C. The sample was microfuged for 10 min, washed with acetone, dried and diluted in distilled water. Protein concentrations of the histone containing supernatant were determined by Bradford protein assay kit (BioRad, Hemel Hempstead, UK).

Western blotting: Immunoprecipitates, whole cell extractions or isolated histones were measured by SDS-PAGE and Western blot analysis using ECL (Amersham, Amersham, UK). Proteins were size-fractionated by SDS-PAGE and transferred to Hybond-ECL membranes. Specific protein bands were detected by ECL according to the manufacturer's instructions.

Immunohistochemistiy: Sequential 12 μm sections were cut from frozen from bronchial biopsies. Sections were fixed in acetone. Biopsies were washed with phosphate buffered saline containing 3% hydrogen peroxide with 0.02% sodium peroxide. Immunostaining was performed using the Vectra Stain kit (Vectra Laboratories, Peterborough, UK). Nonspecific labelling was blocked by coating the plates with normal goat serum for 20 min at room temperature. After washing in PBS the tissues were incubated with a rabbit polyclonal anti-HDAC1 and HDAC2 antibodies (Santa-Cruz, diluted 1:50 in the preincubation solution) at room temperature for 1 hour. After incubation and repeated washing steps with PBS, the sections were subsequently incubated with biotinylated goat anti-rabbit IgG. (1:200) for 1 hr at room temperature. The slides were washed and then avidin-biotin complex was applied for 30 min. Secondary antiserum was detected with a 3, 3′ diaminobenzidine (Sigma, Poole Dorset, UK). Sections were counter-stained with haematoxylin and mounted with mounting medium (DPX). Eosinohils were detected as previously described (Kidney, J et al (1995) Am. J. Respir. Crit. Care Med. 151:1907-1914).

Histone acetylation activity: Cells were plated at a density of 0.25×10⁶ cells/ml and exposed to 0.05 mCi/ml of [³H] acetate (Amersham). After incubation for 10 min at 37° C. cells were stimulated for 6 hr. Histones were isolated and separated by electrophoresis on SDS-16% polyacrylamide gel. Gels were stained with Coommasie brilliant blue and the core histones (H2A, H2B, H3 and H4) excised. The radioactivity in extracted core histones was determined by liquid scintillation counting and normalised to protein level.

Histone deacetylation activity: Radiolabelled histones were prepared from A549 cells following incubation with TSA (100 ng/ml, 6 hr) in the presence of 0.1 mCi/ml [³H]-acetate. Histones were dried and resuspended in distilled water. Crude HDAC preparations were extracted from total cellular homogenates with Tris-based buffer (10 mM Tris-HCl pH 8.0, 500 mM NaCl, 0.25 mM EDTA, 10 mM 2-mercaptoethanol) as previously reported (Kolle, D et al (1998) Methods 15:323-331). The crude HDAC preparation or immunoprecipitates were incubated with [³H]-labelled histone for 30 min at 30° C. before the reaction was stopped by the addition of 1N HCl/0.4N acetic acid. Released [³H]-labelled acetic acid was extracted by ethylacetate and the radioactivity of the supernatant was determined by liquid scintillation counting.

Statistics

Results are expressed as means±standard error of the mean (SEM). A multiple comparison was made between the mean of the control and the means from each individual treatment group by Dunnett's test using SAS/STAT software (SAS Institute Inc., Cary, N.C., USA). All statistical testing was performed using a two-sided 5% level of significance. The concentrations of dexamethasone or trichostatin A producing 50% inhibition (IC₅₀) were calculated from concentration-response curves by linear regression.

Results

Effect of Theophylline on Histone Acetylation and GM-CSF Release in A549 Cells.

In A549 cells lipopolysaccharide (LPS, 3 ng/ml) induced whole cell histone acetyltransferase (HAT) activity at 24 hrs. This was associated with an increase in inflammatory cytokine (GM-CSF) release. Theophylline had a significant concentration-dependent inhibitory effect on LPS-induced whole cell HAT activity although this was not associated with a significant reduction in GM-CSF release (FIG. 10a). Dexamethasone had a far greater inhibitory effect on whole cell HAT activity and a significant inhibitory effect on GM-CSF release (FIG. 10b). Neither theophylline nor dexamethasone had any effect on basasl HAT activity.

Effect of Theophylline on Histone Deacetylation in A549 Cells.

LPS reduced total cell histone deacetylase (HDAC) activity by 30% (give values here) at 6 hrs (FIG. 11a). Theophylline pretreatment (60 min) significantly increased total cell HDAC activity at concentrations up to 10⁻⁵ M measured at 24 hrs. A similar concentration-dependent effect was also seen with dexamethasone (FIG. 11a). In order to investigate the mechanism for this inhibitory effect on HDAC activity we examined the direct effect of theophylline and dexamethasone on nuclear extracts containing HDAC activity as described in the Methods. Theophylline gave a concentration-dependent increase in HDAC activity that reached a maximum at 10⁻⁵ M. At higher concentrations (10⁻⁴-10⁻³ M) theophylline inhibited HDAC activity. In contrast, dexamethasone had no direct effect on HDAC activity (FIG. 11b).

Effect of Theophylline on HDAC Expression

Western blot analysis was used to determine the effect of theophylline and dexamethasone on HDAC expression in A549 cells. Theophylline had no effect on HDAC2 expression. In contrast, theophylline (10⁻⁴ M) induced HDAC1 expression (FIG. 12a) although this effect occurred at a concentration too high to account for the increase in HDAC activity. Indeed, at this concentration theophylline has no effect on total cell HDAC activity (FIG. 11). Dexamethasone increased the expression of both HDAC1 and HDAC2 protein (FIG. 12).

Theophylline Actions on HDAC Activity Do Not Occur Through PDE4 Inhibition or Adenosine Receptor Antagonism.

Theophylline has been proposed to act through PDE4 or through adenosine receptors. We therefore examined the effect of a PDE4 inhibitor (rolipram) and an adenosine receptor antagonist (CGS-15943) on HDAC activity (FIG. 13a). The non-selective PDE inhibitor IBMX (500 μM), rolipram (10 μM) and CGS-15943 (10 μM) had no direct effect on HAT or HDAC activity, indicating that this is a novel molecular action of theophylline. It is also one of the few effects that has been reported at therapeutic drug concentrations. We further investigated the role of MAPK pathways in the theophylline-induced induction of HDAC activity. HDACs are phosphoproteins and alteration in phosphorylation status may markedly affect HDAC activity (Johnson, C. A. & B. M. Turner (1999) Semin. Cell Dev. Biol. 10:179-188). The MEK inhibitor PD089059 (1 μM) failed to have any effect on theophylline-induced increased HDAC activity. In contrast, the p38 MAPK inhibitor SB203580 (1 μM) significantly inhibited theophylline-induced increased HDAC activity (FIG. 13b).

Effect of Theophylline on Glucocorticoid Actions in A549 Cells

We have shown that a major component of glucocorticoid actions in the suppression of inflammatory cytokine production is through recruitment of HDAC activity to the activated transcriptional complex (Example 1). Since theophylline enhances HDAC activity directly we have examined whether theophylline could enhance glucocorticoid activity in vitro in a similar manner to that seen clinically (Evans et al (1997); Ukena, D et al (1997) Eur. Respir. J. 10:2754-2760). Dexamethasone gave a concentration-dependent increase in total cell HDAC activity (FIG. 14). Theophylline (10⁻⁵ M) enhanced the ability of dexamethasone (10⁻¹⁰ M) to increase HDAC activity to levels greater than that seen with 10⁻⁶ M dexamethasone. IL-1β (1 ng/ml) produced a 30-fold increase in GM-CSF release. Low dose theophylline (10⁻⁵ M) and dexamethasone (10⁻¹⁰ M) both caused a 20% decrease in GM-CSF release whereas the combined theophylline/dexamethasone treatment produced a 50% decrease in GM-CSF release. In comparison, dexamethasone (10⁻⁶ M) elicited a 95% decrease in GM-CSF release (FIG. 14).

Effect of Theophylline on HDAC Expression and Activity In Vivo

We examined the effect of 5 weeks treatment with low dose theophylline (Euphylong, 250 μg b.d.) on HDAC activity in 15 mild stable asthmatics using a double blind cross-over controlled study. Blood levels of theophylline were elevated in treated subjects (4.3±0.85) as compared to controls (<1 mg/ml). Clinically there was no significant change in FEV₁ (placebo 3.3±0.16 versus theophylline 3.4±0.09), morning PEF (placebo 456±14 versus theophylline 469±19) or PC₂₀ (placebo 0.98±0.47 versus theophylline 1.2±0.35). However there was a significant reduction in BAL (placebo 3.4±0.47 versus theophylline 1.7±0.28%) and mucosal eosinophils (placebo 1.83±0.48 versus theophylline 1.19±0.43 eosinophils/high powered field).

HDAC1 and 2 was localisation predominantly to the epitheliurm in bronchial biopsies and was not altered by theophylline treatment (FIG. 15). Western blot analysis demonstrated that a significant increase in HDAC1 (0.28±0.09 versus 0.44±0.06, p<0.05) but not HDAC2 (0.28±0.08 versus 0.49±0.20, p=0.1) expression (FIG. 16a, b). In addition there was a significant increase in total HDAC activity in biopsies from subjects treated with theophylline (67±12 versus 111±15 dpm/mg protein, p<0.05)(FIG. 16c).

Effect of Theophylline on HDAC Activity in BAL Macrophages

We examined whether theophylline could also have an effect on HAT and HDAC activity in clinically relevant cells such as BAL macrophages which have been activated during passage through the airway. Macrophages were incubated for 24 hours in the presence of increasing concentrations of theophylline and dexamethasone. Theophylline produced a concentration-dependent increase in HDAC activity that was maximal at 10⁻⁵ M and decreased to control levels at 10⁻⁴ M. In a similar manner, dexamethasone also enhanced HDAC activity in a concentration-dependent manner (FIG. 17a). These increases in HDAC activity were mimicked by alterations in HAT activity (FIG. 17b). Rolipram (10 μM) had no effect on either HAT or HDAC activity.

Discussion

Low concentrations of theophylline within the current therapeutic range had a marked effect on histone acetylation status in A549 cells. Theophylline at concentrations of 10⁻⁶-10⁻⁵M has a significant inhibitory effect on total cell LPS-induced HAT activity. This effect was due to a direct activation of HDAC activity rather than an induction of HDAC expression and may be mediated through ERK MAPK. Inhibitors of the classic theophylline pathways, PDE4s and adenosine receptors, had no effect on the ability of low dose theophylline to induce HDAC activity suggesting that this is a novel target for theophylline action. In contrast, glucocorticoids had no direct effect on HDAC activity but were able to induce total cell HDAC activity via induction of HDAC expression.

Several studies have shown that low doses of theophylline have an anti-inflammatory or immunomodulatory effect in vivo (Brenner, M et al (1988) Clin. Allergy 18:143-150; Finnerty, J. P et al (1996) Eur. Respir. J. 9:1672-1677; Jaffar, Z. H et al (1996) Eur. Respir. J. 9:456-462; Kidney et al (1995); Page, C. P et al (1998) Eur. Respir. J. 12:24-29; Reed, C. E et al (1998) J. Allergy Clin. Immunol. 101:14-23; Shute, J. K et al (1998) Clin. Exp. Allergy 28 Suppl 3:47-52; Sullivan, P et al (1994) Lancet 343:1006-1008; Tinkelman, D. G et al (1993) Pediatrics 92:64-77; Ward, A. J et al (1993) Am. Rev. Respir. Dis. 147:518-523). In our current study we have failed to find any significant effect of theophylline on FEV₁, PC₂₀ and morning PEFR. However we did find a significant decrease in BAL and tissue eosinophils along with an induction of HDAC activity in bronchial biopsies and BAL macrophages. The modest anti-inflammatory effects seen in our patients may be due to the mild nature of the subjects giving little room for improvement.

Several studies have demonstrated an interaction with corticosteroid therapy and the steroid-sparing effects of theophylline (Markham, A. & D. Faulds. (1998) Drugs 56:1081-1091). In patients with moderate and mild asthma addition of low dose theophylline (mean plasma concentration ^{˜}8 mg/L) gave a greater improvement in asthma control than doubling the dose of inhaled corticosteroid (Evans et al (1997); Lim et al (1998); Ukena et al (1997)). These studies suggest that there may be a beneficial interaction between low dose theophylline and corticosteroids. The studies also suggest that theophylline has a molecular mechanism of action that differs from that of corticosteroids. This may be exploited in the control of severe asthma, when addition of theophylline may improve asthma control despite the fact that high doses of inhaled or oral corticosteroid are used (Rivington, R. et al (1995) Am. J. Respir. Crit. Care Med. 151:325-332).

The molecular mechanisms for the anti-inflammatory action of theophylline are unclear. PDE (chiefly PDE3 and PDE4) inhibition in airway smooth muscle can explain theophylline's bronchodilator action (Rabe et al (1995)). However, this action occurs at doses too high to be relevant in our study. Furthermore, theophylline at therapeutic concentrations has no inhibitory effect on PDE in human T-lymphocytes, in contrast to a potent effect of selective PDE4 inhibitors (Giembycz, M. A et al (1996) Br. J. Pharmacol. 118:1945-1958). However, many of the side effects of theophylline, including nausea and headaches, can be ascribed to PDE inhibition, suggesting that if the mechanism of the anti-asthma effect were identified it might be possible to develop safer drugs in the future.

Adenosine is a bronchoconstrictor in asthma and adenosine receptor antagonism by theophylline may occur at therapeutic concentrations. Some of the serious side effects of theophylline, including cardiac arrhythmias and seizures may be due to adenosine receptor antagonism. Although the key adenosine receptor targeted by theophylline in asthma is still uncertain, there is increasing evidence that it might be an A_{2b} receptor on mast cells (Feoktistov, I. & I. Biaggioni (1995) J. Clin. Invest. 96:1979-1986). However, it is unlikely that this mechanism could account for all of the beneficial effects of theophylline in asthma.

More recently it has been shown that low concentrations of theophylline were able to inhibit the activation of NF-κB reduce the expression of inflammatory genes in a manner similar to corticosteroids (Tomita et al (1999)). In addition, eosinophil survival induced by IL-5 and GM-CSF is decreased by low concentrations of theophylline independently from PDE inhibition and changes in cyclic AMP (Ohta et al (1996); Yasui et al (1997)).

We have demonstrated that a major role of glucocorticoids in the repression of inflammatory genes is to recruit HDAC proteins to the site of gene expression (Example 1). This suggests that theophylline should enhance glucocorticoid actions by enabling glucocorticoids to recruit HDACs with increased activity. Indeed in A549 cells we have demonstrated that theophylline synergised with budesonide in enhancing cell HDAC activity and repression of cytokine release. The data from A549 cells suggests that the enhanced HDAC activity seen in the theophylline treated patients would enable low doses of glucocorticoids to have enhanced efficacy in controlling airway inflammation. This is indeed seen in placebo controlled trials (Evans et al (1997); Ukena et al (1997)).

In summary we have shown both in vitro and in vivo theophylline was able to induce a direct activation of HDAC activity. In vitro experiments indicated that this enhanced HDAC activity induced by theophylline was capable of synergising with glucocorticoids on increasing total cell HDAC activity and GM-CSF release. This suggests that the molecular mechanism behind the increased additive effect of theophylline on glucocorticoid actions in vivo is related to increased HDAC activity being recruited by GR to suppress inflammatory genes. This also indicates why theophylline on its own is not a particularly efficient anti-inflammatory agent. Without the presence of GR the activated HDAC is not targeted to the site of inflammatory gene transcription. These studies suggest that there is a potential to develop novel therapeutic agents with improved anti-inflammatory properties to use as steroid add on therapies which have improved HDAC activation properties and reduced PDE4 profile.

### EXAMPLE 3

BDAC Assay for Theophylline-Like Compounds.

Histone deacetylation assays may be set up with standard amounts of radiolabelled histones prepared from cultured cells following incubation of the cells with trichostatin A (TSA, 100 ng/ml, 6 hr) in the presence of 0.1 mCi/ml [³H]-acetate. The assay will contain either standard amounts of crude HDAC activity isolated from cultured cells, immunoprecipitated HDAC proteins or purified cloned HDAC proteins.

The HDAC preparations are incubated with [³H]-labelled histone for 30 min at 30° C. before the reaction is stopped by the addition of 1N HCl/0.4N acetic acid. [³H]-labelled acetic acid is released from the histone preparation and extracted by ethylacetate and the radioactivity of the supernatant determined by liquid scintillation counting.

The concentration-dependent effect of theophylline-like compounds to modulate the activity of the crude HDAC preparations, purified HDACs or cloned HDACs is determined by comparison with control compounds including theophylline.

### EXAMPLE 4

Effect of Theophylline Under Conditions of Oxidative Stress

We show that theophylline can enhance dexamethasone actions under conditions of oxidative stress where dexamethasone is only weakly effective. This may be very important in severe asthma and COPD where steroids are clinically not effective at doses that do not produce side-effects. Thus theophylline may be steroid-sparing and enhance steroid-responsiveness in these types of patients.

In alveolar macrophages from non-smokers we found that theophylline (10⁻⁵ M) significantly enhanced HDAC activity whereas dexamethasone (10⁻¹⁰ M) had no direct effect. Combined treatment with dexamethasone (10⁻¹⁰ M) and theophylline (10⁻⁵ M) markedly enhanced the effect seen with theophylline alone. This effect was similar that seen with 10⁻⁶ M dexamethasone (FIG. 20a). These results correlated with functional repression of LPS-induced IL-8 release by combined theophylline and dexamethasone (FIG. 20b). Low concentrations of theophylline alone had no effect on LPS-induced IL-8 release, presumably, as the increased HDAC activity is not targeted to the activated transcriptional complex.

Macrophages obtained from smokers had a much-reduced level of HDAC activity that was not affected by dexamethasone alone even at high concentrations (10⁻⁶ M). Theophylline enhanced HDAC activity as in non-smokers and this was further enhanced following combination treatment. The results on HDAC activity correlated with suppression of IL-8 release (FIG. 20a). Reduced HDAC activity in macrophages from smokers correlated also with the greater induction of IL-8 seen in these cells (FIG. 20b).

Effect of Theophylline on Histone Deacetylase Activity and Expression and Cytokine Production in IL-1β L plus H₂O₂ stimulated A549 cells.

Since we observed that alveolar macrophages obtained from smokers had reduced HDAC activity we investigated whether theophylline could reverse oxidant stress-induced inhibition of HAT and HDAC activity. Combination of IL-1β and H₂O₂ increased total cell HAT activity at 4 hrs. Theophylline alone (10⁻⁵ M) had no effect on total cell HAT activity whereas dexamethasone alone (10⁻⁶ M) inhibited the IL-1β plus H₂O₂-dependent increase in HAT activity completely (FIG. 21a). HDAC activity was markedly reduced by IL-1β plus H₂O₂ treatment by 35% (83±4 versus 131±10 dpm) at 4 hrs (FIG. 21b). Theophylline pre-treatment (10⁻⁵ M, 10 min) significantly increased total cell HDAC activity whereas dexamethasone had no effect on HDAC activity at this time point (FIG. 21b).

Theophylline incubation for 24 hrs had no effect on HDAC1 and 2 expression. However, theophylline restored IL-1β+H₂O₂-induced reduction in HDAC2 expression. Dexanmethasone was able to induce HDAC1 and HDAC2 expression (FIGS. 21c, d) under both conditions.

Theophylline Targets HDAC1 and 3.

Using IP-HAT assays we were able to determine which HDACs were the target for theophylline action. Theophylline (10⁻⁵ M) caused a significant induction of both HDAC1 and HDAC3 activity without having any effect on HDAC2 activity (FIG. 22).

Effect of Combination of Low Dose Theophylline and Low Dose Dexamethasone on HDAC Activity and GM-CSF Production in A549 Cells

As described above for alveolar macrophages we investigated the effect of theophylline on dexamethasone actions in A549 cells. Treatment of cells with IL-1β plus H₂O₂ reduced HDAC activity (83±4 versus 132±11 dpm/μg protein). Theophylline (10⁻5 M) restored total HDAC activity back to control levels (130±3 versus 83±4 dpm/μg protein) whereas dexamethasone (10⁻¹⁰ and 10⁻⁶ M) had no effect. Combination of theophylline (10⁻⁵ M) and dexamethasone (10⁻¹⁰ M) also increased total HDAC activity (139±9 dpm/μg protein) (FIG. 23a). Functionally both theophylline (10⁻⁵ M) and dexamethasone (10⁻10 M) alone failed to inhibited IL-1β-stimulated GM-CSF release. Combined treatment caused a 70% repression of GM-CSF release (229±84 versus 714±94 pg/ml) which was blocked by pre-treatment with the HDAC inhibitor trichostatin A (TSA, FIG. 23). Dexamethasone (10⁻⁶ M) alone caused a 96% inhibition of GM-CSF release which was inhibited by 48% by TSA (506±40 versus 969±84 pg/ml). H₂O₂ further enhanced IL-1β -stimulated GM-CSF release (2054±342 versus 714±94 pg/ml)(FIG. 23b & c). Again neither theophylline (10⁻⁵ M) nor dexamethasone (10⁻¹⁰ M) alone had any effect on GM-CSF release. Combined treatment suppressed GM-CSF release by 71% (623±180 versus 2054±352 pg/ml) an effect that was blocked by TSA. In comparison, dexamethasone (10⁻⁶ M) suppression of GM-CSF release was reduced compared to that seen after IL-1β-stimulation alone (46% versus 96% inhibition). TSA was unable to block this effect suggesting that H₂O₂ was targeting HDAC activity.

1. A screening method for identifying a drug-like compound or lead compound for the development of a drug-like compound in which (1) a xanthine or related compound is exposed to a histone deacetylase, (2) the binding of the compound to the histone deacetylase is measured or the change in the activity of the histone deacetylase is measured or the change in the binding of the histone deacetylase to activated glucocorticoid receptor (GR) is measured and (3) any compound capable of the required binding to the histone deacetylase or producing the required change in the activity of the histone deacetylase or its binding to activate glucocorticoid receptor is identified.

2. A screening method for identifying a drug-like compound or lead compound for the development of a drug-like compound wherein the ability of a xanthine or related compound to modulate the expression of a histone deacetylase gene, or expression from a transcriptional regulatyr sequence derived from a histone deacetylase gene, is measured and any compound capable of effecting the required modulation in the expression of the said histone deacetylase gene, or in the expression from the said transcriptional regulatory sequence, is identified.

3. A method for modulating a histone deacetylase activity wherein the histone deacetylase is exposed to a compound identified or identifiable by the method of claim 1.

4. Use of a compound identified or identifiable by the method of claim 1 in a method for modulating a histone deacetylase activity wherein the histone deacetylase is exposed to a compound identified or identifiable by the method of claim 1.

5. The use or method of any of the preceding claims wherein the xanthine is a methylxanthine.

6. The use or method of claim 5 wherein the methylxanthine is theophylline or a salt thereof.

7. The use or method of any one of claims 1 to 6 performed in vitro.

8. The use or method of any one of claims 1 to 7 wherein the histone deactetylase is or comprises histone deacetylase 1, histone deacetylase 2 and/or histone deacetylase 3.

9. The method of any of claims 1, 2 or 5 to 8 fiber comprising the steps of (1) exposing the compound to a phosphodiesterase activity and determining the effect of the compound on the phosphodiesterase activity and/or (2) exposing the compound to an adenosine receptor and determining the activity of the compound as an adenosine receptor antagonist and (3) any compound capable of the required effect on phosphodiesterase activity and/or having the required activity as an adenosine receptor antagonist is identified.

10. The method of any of claims 1, 2 or 5 to 9 wherein the required change in the activity of the histone deacetylase is an increase in the said activity or wherein the required change in the binding of the histone deacetylase to activated glucocorticoid receptor (GR) is an increase in the said binding.

11. The method of any of claims 1, 2 or 5 to 10 wherein the xanthine or related compound is exposed to a histone deacetylase or the ability of a xanthine or related compound to modulate the expression of a histone deacetylase is measured in the presence of a glucocorticoid.

12. A compound identifiable by the screening method of any one of claims 1, 2 or 5 to 11 wherein the compound is not theophylline, caffeine, acepifylline, bamifylline, bufylline, cafaminol, cafedrine, diprophylline, doxofylline, enprofylline, etamiphylline, etofylline, proxyphylline, suxamidofylline, theobromine or a salt thereof, or a glucocorticoid or pyridinylimidazole compound.

13. The compound of claim 12 for use in medicine.

14. Use of a compound identifiable by the screening method of any one of claims 1, 2 or 5 to 11 in the manufacture of a medicament for the treatment of a patient in need of modulation of histone deacetylase activity, wherein the patient is not in need of modulation of histone deacetylase activity on account of having asthma or other inflammatory airway disease.

15. Use of a compound identifiable by the screening method of claim 10 in the manufacture of a medicament for the treatment of a patient in need of an increase in histone deacetylase activity or a decrease in histone acetylation, wherein the patient is not in need of modulation of histone deacetylase activity on account of having asthma or other inflammatory airway disease.

16. Use of a compound according to claim 12 in the manufacture of a medicament for the treatment of a patient with asthma or other airway disease or other chronic inflammatory disease.

17. Use of a compound identifiable by the screening method of any of claims 1, 2 or 5 to 11 in the manufacture of a medicament for the treatment of a disorder of cellular differentiation and/or proliferation in which excessive phosphodiesterase 3 or 4 activity or excessive adenosine receptor activity have not been implicated, but in which histone deacetylase or the level of histone acetylation has been implicated in causing or exacerbating the disorder.

18. A method of treatment of a patient in need of modulation of histone deacetylase activity, comprising administering an effective amount of a compound identified or identifiable by the screening method of any one of claims 1, 2 or 5 to 11, wherein the patient is not in need of modulation of histone deacetylase activity on account of having asthma or other inflammatory airway disease.

19. A method of treatment of a patient in need of an increase in histone deacetylase activity or a decrease in histone acetylation, comprising administering an effective amount of a compound identified or identifiable by the screening method of claim 10, wherein the patient is not in need of modulation of histone deacetylase activity on account of having asthma or other inflammatory airway disease.

20. A method of treatment of a patient with asthma or other inflammatory airway disease, comprising administering an effective amount of a compound according to claim 12.

21. A method of treatment of a patient in need of modulation of histone deacetylase or histone acetylation, or with a disorder of cellular differentiation and/or proliferation in which excessive phosphodiesterase 3 or 4 activity or excessive adenosine receptor activity have not been implicated, but in which histone deacetylase or the level of histone acetylation has been implicated in causing or exacerbating the condition, comprising administering an effective amount of a compound identifiable by the screening method of any of claims 1, 2 or 5 to 11.

22. The use or method of any of claims 14 to 21 wherein a glucocorticoid is, has been, or will be administered to the patient.

23. A kit of parts comprising a glucocorticoid and a compound according to claim 12.

24. A kit of parts suitable for carrying out a method according to any one of claims 1, 2 or 5 to 11 comprising a histone deacetylase, a xanthine or related compound.

25. A kit of parts according to claim 24 further comprising a glucocorticoid.

26. Use of a histone deacetylase in a method of identifying an anti-asthmatic agent.

27. A screening method for identifying a drug-like compound or lead compound for the development of a drug-like compound for treating asthma or other inflammatory airway disease in which (1) a test compound is exposed to a histone deacetylase, (2) the binding of the compound to the histone deacetylase is measured or the change in the activity of the histone deacetylase is measured or the change in the binding of the histone deacetylase to activated glucocorticoid receptor (GR) is measured and (3) any compound capable of the required binding to the histone deacetylase or producing the required change in the activity of the histone deacetylase or its binding to activated glucocorticoid receptor is identified.

28. A screening method for identifying a drug-like compound or lead compound for the development of a drug-like compound for treating asthma or other inflammatory airway disease, ulcerative colitis and/or rheumatoid arthritis, wherein the ability of a test compound to modulate the expression of a histone deacetylase gene, or expression from a transcriptional regulatory sequence derived from a histone deacetylase gene, is measured and any compound capable of effecting the required modulation in the expression of the said histone deacetylase gene, or in the expression from the said transcriptional regulatory sequence, is identified.

29. Use of a compound which increases histone deacetylase activity in the manufacture of a medicament for treatment of a patient with asthma or other inflammatory airway disease, wherein the compound is not theophylline, caffeine, acepifylline, bamifylline, bufylline, cafaminol, cafedrine, diprophylline, doxofylline, enprofylline, etamiphylline, etofylline, proxyphylline, suxamidofylline, theobromine or a salt thereof, or a glucocorticoid or pyridinylimidazole compound.

30. A method of treatment of a patient with asthma or other inflammatory airway disease comprising administering an effective amount of a compound which increases histone deacetylase activity, wherein the compound is not theophylline, caffeine, acepifylline, bamifylline, bufylline, cafaminol, cafedrine, diprophylline, doxofylline, enprofylline, etamiphylline, etofylline, proxyphylline, suxamidofylline, theobromine or a salt thereof, or a glucocorticoid or pyridinylimidazole compound.

31. A composition comprising a compound according to claim 12 and a pharmaceutically acceptable excipient.
